(19) United States
(12) Patent Application Publication
(10) Pub. No.: US 2026/0308201 A1
(43) Pub. Date: Oct. 8, 2026

(54) THERAPEUTIC PEPTIDES

(71) Applicants: Shape Therapeutics Inc., Seattle, WA (US); The Regents of the University of California, Oakland, CA (US)

(72) Inventors: Prashant Mali, La Jolla, CA (US); Sami Nourreddine, La Jolla, CA (US); Daihua Chen, La Jolla, CA (US); Rafael Ponce, Seattle, WA (US)

(21) Appl. No.: 19/108,237

(22) PCT Filed: Sep. 7, 2023
(86) PCT No.: PCT/US2023/073613

§ 371 Date: Mar. 3, 2025

Related U.S. Application Data
(60) Provisional application No. 63/404,990, filed on Sep. 9, 2022.

(51) Int. Cl. C07K 7/08; A61K 38/00; A61P 35/00

(52) U.S. Cl. CPC C07K 7/08; A61P 35/00; A61K 38/00

(57) ABSTRACT

Disclosed herein are engineered peptides for treatment of diseases or conditions such as cancer. Also disclosed herein are pharmaceutical compositions containing engineered peptides of the present disclosure. Also disclosed herein are methods of treating a disease or condition by administering an engineered peptide of the present disclosure. Also disclosed herein are methods of making engineered peptides described herein.

### CROSS-REFERENCE

This application claims the benefit of U.S. Provisional Application No. 63/404,990, filed Sep. 9, 2022, the disclosure of which is incorporated herein by reference in its entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

In some aspects, an engineered peptide has at least about 84% sequence identity to any one of SEQ ID NO: 1, SEQ ID NO: 2, or SEQ ID NO: 3. In some embodiments, the engineered peptide has a length of from about 10 amino acids to about 40 amino acids or of from about 10 amino acids to 39 amino acids. In some embodiments, the engineered peptide has a length of at most 15 amino acids. In some embodiments, the engineered peptide has a length of 13 amino acids or 14 amino acids. In some embodiments, the engineered peptide has the amino acid sequence of any one of SEQ ID NO: 1, SEQ ID NO: 2, or SEQ ID NO: 3. In some embodiments, the engineered peptide adopts an alpha helical structure at physiological salt concentrations and physiological temperature. In some embodiments, the engineered peptide is a stapled peptide In some embodiments, the stapled peptide is an (i, i+3) stapled peptide or an (i, i+7) stapled peptide. In some embodiments, the stapled peptide is an (i, i+7) stapled peptide. In some embodiments, the (i) residue of the stapled peptide is R-octenylalanine (R8), S-octenylalanine (S8), R-pentylalanine (R5), or S-pentenylalanine (S5). In some embodiments, the (i+3) residue or the (i+7) residue of the stapled peptide is R-octenylalanine (R8), S-octenylalanine (S8), R-pentylalanine (R5), or S-pentenylalanine (S5). In some embodiments, the (i) residue of the stapled peptide is at amino acid 3 or amino acid 4 of any one of SEQ ID NO: 1, SEQ ID NO: 2, or SEQ ID NO: 3 In some embodiments, the (i) residue of the stapled peptide is R-octenylalanine (R8) and the (i+3) residue of the stapled peptide is S-pentenylalanine (S5). In some embodiments, the (i) residue of the stapled peptide is R-octenylalanine (R8) and the (i+7) residue of the stapled peptide is S-pentenylalanine (S5). In some embodiments, the engineered peptide has at least 84% sequence identity to any one of SEQ ID NO: 4, SEQ ID NO: 5, or SEQ ID NO: 6. In some embodiments, the engineered peptide has the amino acid sequence of any one of SEQ ID NO: 4, SEQ ID NO: 5, or SEQ ID NO: 6. In some embodiments, the engineered peptide has an IC₅₀ of from about 1 nM to about 50 μM against a cancer cell line, as determined in an in vitro assay.

In some aspects, an engineered peptide comprises (a) at least 80%, at least 85%, at least 90%, at least 95%, or at least 99% sequence identity to a contiguous portion of an RB1 peptide, a CHEK2 peptide, an MDM2 peptide, a RAF1 peptide, an RRAS2 peptide, a RAC1 peptide, a PIK3Ca peptide, a PIK3R1 peptide, a VHL peptide, a DDX3X peptide, or a CTNNB1 peptide; and (b) a length of from about 10 amino acids to about 20 amino acids; wherein the engineered peptide has an IC₅₀ of from about 1 nM to about 50 M against a cancer cell line, as determined in an in vitro assay. In some embodiments, the engineered peptide is a salt of an engineered peptide. In some embodiments, the salt of the engineered peptide is an acetate salt or a trifluoroacetate (TFA) salt. In some embodiments, the engineered peptide is a stapled peptide. In some embodiments, the stapled peptide is an (i, i+3) stapled peptide or an (i, i+7) stapled peptide. In some embodiments, the stapled peptide is an (i, i+7) stapled peptide.

In some aspects, a pharmaceutical composition that comprises the engineered peptide of any one of the embodiments described herein and a pharmaceutically acceptable excipient, diluent, or carrier. In some embodiments, the pharmaceutical composition is in unit dose form. In some embodiments, the engineered peptide in unit dose form is about 5 mg/kg, about 15 mg/kg, about 50 mg/kg, about 55 mg/kg, about 60 mg/kg, about 65 mg/kg, about 75 mg/kg, about 100 mg/kg, about 150 mg/kg, about 200 mg/kg, about 240 mg/kg, or about 300 mg/kg.

In some aspects, a method of treating a disease or condition in a subject in need thereof, the method comprises administering to the subject the engineered peptide of any one of the embodiments disclosed herein or the pharmaceutical composition of any one of the embodiments disclosed herein, wherein the administering is sufficient to treat the disease or condition. In some embodiments, the disease or condition is a cancer. In some embodiments, the cancer comprises a KRAS mutation. In some embodiments, the KRAS mutation comprises a G12V mutation, a G12C mutation, or a G12D mutation of KRAS. In some embodiments, the cancer is a melanoma, a colorectal cancer, a pancreatic cancer, a bladder cancer, a breast cancer, a triple negative breast cancer, an ovarian cancer, a juvenile myelomonocytic leukemia (JMML), a hematologic malignancy, a cholangiocarcinoma, a uterine cancer, a cervical cancer, a testicular cancer, a lung cancer, or any combination thereof. In some embodiments, the administering is parenteral or intra-tumoral. In some embodiments, the subject in need thereof is a mammal. In some embodiments, the mammal is a human, non-human primate, dog, cat, hamster, guinea pig, rat, or mouse. In some embodiments, the method further comprises administering a second therapy to the subject. In some embodiments, the second therapy comprises a chemotherapy, a radiation therapy, a hormone therapy, a hyperthermia therapy, an immunotherapy, a photodynamic therapy, a stem cell therapy, a surgery, a targeted therapy, or any combination thereof. In some embodiments, the second therapy is administered concurrently or consecutively with the engineered peptide or the pharmaceutical composition.

In some aspects, a method of making engineered therapeutic peptides for treatment of cancer comprises (a) selecting portions of a candidate polypeptide that display cellular toxicity, as determined in an in vitro assay; (b) curating the portions of the candidate polypeptides to select a subset of the portions of the candidate polypeptide that adopt an alpha helical structure, as determined by x-ray crystallography; (c) modifying the subset using mutagenesis to generate engineered therapeutic peptides, and (d) selecting engineered therapeutic peptides having an IC₅₀ of from about 1 nM to about 50 μM against a cancer cell line, as determined in an in vitro assay. In some embodiments, the modifying of (c) further comprising making an (i, i+7) staple within the engineered therapeutic peptides.

In some aspects, a method of inhibiting cell proliferation in a cancerous cell comprises administering to the cancerous cell the engineered peptide of any one of the embodiments disclosed herein or the pharmaceutical composition of any one of the embodiments disclosed herein, wherein the administering is sufficient to inhibit proliferation of the cancerous cell.

### BRIEF DESCRIPTION OF THE DRAWINGS

Novel features of exemplary embodiments are set forth with particularity in the appended claims. A better understanding of the features and advantages will be obtained by reference to the following detailed description that sets forth illustrative embodiments, in which the principles of the disclosed systems and methods are utilized, and the accompanying drawings of which:

FIG. 1 depicts peptide fragments count distribution for fitness score and Log 2FC values after filtering for alpha helical regions, crystal structure availability and interacting residue inclusion. Each count is a 40-mer peptide from the fitness screen that meets the filtering requirements.

FIG. 2 depicts a workflow diagram for in silico identification of mutations that improve binding affinity between a 40-mer peptide and an interacting protein, and subsequent screening.

FIG. 3A depicts a heatmap of computational alanine scanning results of identified alpha helical 40-mer peptides (5 out of 11) binding to interacting proteins. The Protein Data Bank Identifier (PDB ID) followed by the interacting protein is labeled on the right side. The amino acid sequence of the 40-mer peptide is labeled on the bottom and the corresponding computational alanine binding energy with the interacting protein, delta-delta-G (ddG), is plotted. The higher the value is, the more likely that particular residue is important for binding.

FIG. 3B is a heatmap of the point mutation scanning result of RAF1_78 binding to RAP1A (PDB ID: 1C1Y). The residues for conducting site saturation mutagenesis are identified via the results from computational scanning and is labelled on the bottom. The mutated amino acids are labelled to the left and the ddG of the final mutated structure is plotted. The more negative the value is, the higher the binding affinity is between the 40-mer peptide-interacting protein complex. The circled residue is A85K and has been identified as increasing binding affinity between RAF1 and HRAS.

FIG. 4 depicts the structure of the interaction between RAF1 and interacting proteins (RAP1A (top) and HRAS (bottom)).

FIGS. 5A and 5B are zoomed in structures of the interaction between RAF1 and interacting proteins (RAP1A (top) and HRAS (bottom)) (FIG. 5A) or RAC1 and interacting proteins (PREX1 (top), DOCK2 (middle), and PLXNB1 (bottom)) (FIG. 5B).

FIG. 6A-6B shows dose response curves of engineered peptides (SEQ ID NO: 4 peptide, SEQ ID NO: 5 peptide, or SEQ ID NO: 6 peptide) on MDA-MB-231 or A375 cell survival at 24-hours (FIG. 6A) and 48-hours (FIG. 6B) post-treatment. TAT-FLAG is a negative control.

FIG. 7A-7B depict starvation potentiates the anti-proliferative effect of the engineered peptide, SEQ ID NO: 4 peptide. FIG. 7A shows A375 cells that were treated for 48-hours with SEQ ID NO: 4 peptide at 10 μM or vehicle (PBS) with either 10% FBS or 0% FBS (starvation condition). Bright field images were taken with 20× magnification objective. Scale bar=100 μm.

FIG. 7B shows quantification of cell viability measured by the colorimetric assay Cell Counting Kit-8 (CCK8).

FIGS. 8A-8B show tumor experiment timeline and results. FIG. 8A shows a timeline for tumor experiments. FIG. 8B depicts the change in tumor volume observed after injection of SEQ ID NO: 4 peptide or a Flag control.

FIGS. 9A-9C show tumor experiment timeline and results. FIG. 9A shows a timeline for tumor experiments. FIG. 9B depicts the change in tumor volume observed after injection of SEQ ID NO: 4 peptide or a Flag control. FIG. 9C shows tumor size after the day of injection (Day 7) and the day after injection (Day 8).

FIGS. 10A-10B show tumor experiment timeline and results. FIG. 10A shows a timeline for tumor experiments. FIG. 10B depicts the change in tumor volume observed after injection of SEQ ID NO: 4 peptide (15 mg/kg, 10 mg/kg, or 5 mg/kg) or a Flag control (15 mg/kg).

FIGS. 11A-11C show Half Maximal Inhibitory Concentration (IC50) curves for SEQ ID NO: 4 and a control peptide (TAT-Flag peptide) against different KRAS-mutant cell lines. FIG. 11A shows IC50 curves for the control peptide (top) or SEQ ID NO: 4 peptide (bottom) for the H358 cell line; FIG. 11B shows IC50 curves for the control peptide (top) or SEQ ID NO: 4 peptide (bottom) for the H441 cell line; and FIG. 11C shows IC50 curves for the control peptide (top) and SEQ ID NO: 4 peptide (bottom) for the SW480 cell line.

FIGS. 12A-12B show the tumor weight from necropsy of animals after 4 weeks of treatment. FIG. 12A shows the grouped data from the vehicle group, the control peptide (TAT-FLAG) group, the SEQ ID NO: 4 peptide low dose group, and the SEQ ID NO: 4 peptide high dose group. FIG. 12B shows the individual data from the vehicle group, the control peptide (TAT-FLAG) group, the SEQ ID NO: 4 peptide low dose group, and the SEQ ID NO: 4 peptide high dose group. On both graphs, the tumor weight is shown on the Y-axis, the tumor group is indicated on the X-axis, and the number of animals per group is indicated below the X axis. From left to right, the data is presented as the vehicle group, the control peptide (TAT-FLAG) group, the SEQ ID NO: 4 peptide low dose group, and the SEQ ID NO: 4 peptide high dose group.

FIGS. 13A-13C show the mean tumor volumes for the SW480 model, the NCI-H358 model, and the NCI-H441 model. FIG. 13A shows the mean tumor volume for the SW480 model. FIG. 13B shows the mean tumor volume for the NCI-H358 model. FIG. 13C shows the mean tumor volume for the NCI-H441 model. On all graphs, the tumor volume (mm³) is shown on the Y-axis, and the days post start of dosing is indicated on the X-axis. The tumor volume was measured with a caliper.

FIGS. 14A-14C show tumor volume by tumor weight correlations for the SW480 model, the NCI-H358 model, and the NCI-H441 model. FIG. 14A shows the tumor volume by tumor weight correlation for the SW480 model. FIG. 14B shows the tumor volume by tumor weight correlation for the NCI-H358 model. FIG. 14C shows the tumor volume by tumor weight correlation for the NCI-H441 model. In all figures, the tumor volume is shown on the Y-axis and the tumor weight is shown on the X-axis. G1 is the vehicle group, G2 is the control TAT-Flag peptide group, G3 is the SEQ ID NO: 4 peptide low dose group, and G4 is the SEQ ID NO: 4 peptide high dose group.

FIGS. 15A-15C show the mean body weights of mice for the SW480 model, the NCI-H358 model, and the NCI-H441 model. FIG. 15A shows the mean body weight for the SW480 model. FIG. 15B shows the mean body weight for the NCI-H358 model. FIG. 15C shows the mean body weight for the NCI-H441 model. On all graphs, percent body weight change is shown on the Y-axis, and the days from the start of the dosing is indicated on the X-axis.

### DETAILED DESCRIPTION

### Overview

Disclosed herein are engineered peptides, methods of screening for engineered peptides, and methods of treating diseases or conditions utilizing engineered peptides. An engineered peptide of the present disclosure can be a therapeutic peptide and can be selected to exhibit cellular toxicity upon administration, which can be useful for treating diseases such as cancer.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “substantially” or “essentially” refers to a qualitative condition that exhibits an entire or nearly total range or degree of a feature or characteristic of interest. In some cases, substantially refers to at least about: 70%, 75%, 80%, 85%, 90%, 95%, 99%, 99.9% or 99.99% of the total range or degree of a feature or characteristic of interest. In some cases, the substantially or essentially refers to an amount that can be about 100% of a total amount.

As used herein, the term “about” a number can refer to that number plus or minus 10% of that number.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows: [(Number of Identical Positions)/(Total Number of Positions in the Test Sequence)]×100%

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023 are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows: Percent Sequence Identity=(“Percent Identity” output value)×(“Query Coverage” output value)

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (SEQ ID NO: 11) (length=10 nucleotides) to reference sequence 2: AAAAAAAAAA (SEQ ID NO: 12) (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (SEQ ID NO: 13) (length=20 nucleotides) to reference sequence 4: GGGGGGGGGG (SEQ ID NO: 14) (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (SEQ ID NO: 14) (length=10 nucleotides) to reference sequence 6: CCCCCGGGGGGGGGGCCCCC (SEQ ID NO: 13) (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (SEQ ID NO: 15) (length=10 amino acids) to reference sequence 8: YYYYYYYYYY (SEQ ID NO: 16) (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (SEQ ID NO: 17) (length=20 amino acids) to reference sequence 10: FFFFFYYYYY (SEQ ID NO: 15) (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (SEQ ID NO: 15) (length=10 amino acids) to reference sequence 12: LLLLLFFFFFYYYYYLLLLL (SEQ ID NO: 17) (length=20 amino acids). The percent identity between test sequence 11 and reference sequence 12 would be [(10)/(10)]×100%=100%. Test sequence 11 has 100% sequence identity to reference sequence 12.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “in vivo” refers to an event that takes place in a subject's body.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids. A sequence of a polynucleotide may be provided interchangeably as an RNA sequence (containing U) or a DNA sequence (containing T). A sequence provided as an RNA sequence is intended to also cover the corresponding DNA sequence and the reverse complement RNA sequence or DNA sequence. A sequence provided as a DNA sequence is intended to also cover the corresponding RNA sequence and the reverse complement RNA sequence or DNA sequence.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together-optionally to preserve the conformation of the fused protein domains, prevent unfavorable interactions between the fused protein domains which can compromise their respective functions, or both.

As used herein, “pharmaceutically acceptable salt” may refer to pharmaceutical molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### Engineered Peptides

Disclosed herein are engineered peptides useful for treatment of a disease or condition in a subject. An engineered peptide as disclosed herein can be a therapeutic peptide. As disclosed herein, a therapeutic peptide exhibits a therapeutic property useful for treatment of a disease or condition. In some instances, the disease or condition is a cancer.

In some embodiments, an engineered peptide of the present disclosure is a therapeutic peptide that exhibits cellular toxicity. As disclosed herein, an engineered peptide of the present disclosure is a therapeutic peptide exhibiting cellular toxicity can be useful to treat diseases or conditions such as cancer. In some instances, contacting an engineered peptide of the present disclosure with a cancer cell can arrest cancer cell growth. In some instances, contacting an engineered of the present disclosure can reduce the size of a cancer cell (e.g. can decrease tumor volume). In some instances, contacting an engineered of the present disclosure can induce apoptosis or cell death of the cancer cell.

In some embodiments, an engineered peptide of the present disclosure can be alpha-helical (e.g. adopts an alpha-helical structure at physiological salt concentrations and physiological temperature). In some cases, a physiological temperature can be from about 36° C. to about 38° C., or about 37° C. degrees. In some cases, a physiological salt concentration can be from about 0.5% to about 15%, 1% to about 10%, 1% to about 5%, or about 0.9%, 1%, 2%, 3%, 4%, 5%, 6%, 7%, 8%, 9%, 10% of a solution. In some cases, a physiological salt can be a sodium chloride, a sodium phosphate, a potassium phosphate, a sodium carbonate, a potassium iodide, or any combination thereof. Without withing to be bound by theory, an alpha-helical engineered peptide of the present disclosure may have improved cell permeability, relative to a corresponding non-alpha helical peptide. As such, an alpha-helical engineered peptide of the present disclosure is useful for producing cellular toxicity to treat diseases or conditions described herein.

In some embodiments, an engineered peptide of the present disclosure can be a stapled peptide. In some embodiments, an alpha-helical engineered peptide of the present disclosure can be a stapled peptide. A stapled peptide of the present disclosure is a peptide in which two or more amino acids of the peptide are covalently linked by way of their side chains. Without wishing to be bound by theory, utilizing peptide stapling can stabilize a secondary structure adopted by the stapled peptide. Where the engineered peptide is an alpha helical engineered peptide, utilizing peptide stapling to produce a stapled alpha helical engineered peptide can stabilize the alpha helical structure of the engineered peptide, thus reducing the entropic penalty for adopting the alpha helical secondary structure. In some cases, a peptide herein can comprise one or more staples. In some cases, a stapled peptide encompasses a stitched peptide having, for example, a plurality of staples (e.g., 2, 3, 4, 5, 6, or 7 staples). A stitched peptide contains 2 or more staples. In some cases, a first residue (i) can be stapled to a residue that is n residues downstream (i+n). For example, a first residue (i) can be stapled to a residue 3 amino acids downstream (i+3), 4 amino acids downstream (i+4), 5 amino acids downstream (i+5), 6 amino acids downstream (i+6), 7 amino acids downstream (i+7), 8 amino acids downstream (i+8), 9 amino acids downstream (i+9), 10 amino acids downstream (i+10), 11 amino acids downstream (i+11), 12 amino acids downstream (i+12), 13 amino acids downstream (i+13), 14 amino acids downstream (i+14), etc. In some embodiments, a therapeutic peptide of the present disclosure is an (i, i+3) stapled peptide. In some embodiments, an engineered peptide of the present disclosure is an (i, i+7) stapled peptide. The first residue (i) in a peptide staple can be any residue that is at least n upstream of the last residue an engineered peptide as disclosed herein. For example, in an engineered peptide having a length of 13 amino acids and comprising a first residue (i) stapled to a residue n amino acids downstream (i+n) wherein n is 7, the first residue (i) can be amino acid 1, 2, 3, 4, 5, or 6 of the engineered peptide. In some embodiments, an engineered peptide having a length of 13 amino acids and a stapled peptide at (i, i+7) comprises a first residue (i) at amino acid 3 of the engineered peptide that is stapled to residue (i+7) at amino acid 10 of the engineered peptide. For example, in an engineered peptide having a length of 14 amino acids and comprising a first residue (i) stapled to a residue n amino acids downstream (i+n) wherein n is 7, the first residue (i) can be amino acid 1, 2, 3, 4, 5, 6, or 7 of the engineered peptide. In some embodiments, an engineered peptide having a length of 14 amino acids and a stapled peptide at (i, i+7) comprises a first residue (i) at amino acid 4 of the engineered peptide that is stapled to residue (i+7) at amino acid 11 of the engineered peptide. As another example, in an engineered peptide having a length of 13 amino acids and comprising a first residue (i) stapled to a residue n amino acids downstream (i+n) wherein n is 3, the first residue (i) can be amino acid 1, 2, 3, 4, 5, 6, 7, 8, 9, or 10 of the engineered peptide. As another example, in an engineered peptide having a length of 14 amino acids and comprising a first residue (i) stapled to a residue n amino acids downstream (i+n) wherein n is 3, the first residue (i) can be amino acid 1, 2, 3, 4, 5, 6, 7, 8, 9, 10, or 11 of the engineered peptide. In some embodiments, a (i) residue is a non-natural amino acid. In some embodiments, a (i+n) residue that is n amino acids downstream of the (i) residue is a non-natural amino acid. In some embodiments, the (i) residue is an α, α-disubstituted non-natural amino acid. In some embodiments, the (i) residue is S-pentenylalanine (S5), R-pentylalanine (R5), S-octenylalanine (S8), or R-octenylalanine (R8). In some embodiments, the (i+n) residue is S-pentenylalanine (S5), R-pentylalanine (R5), S-octenylalanine (S8), or R-octenylalanine (R8). For example, in an engineered peptide comprising an (i) residue stapled to an (i+n) residue, the residue (i) can be R8 and the (i+n) residue can be S5.

Engineered peptides of the present disclosure can be engineered or non-naturally occurring peptides. As disclosed herein, an engineered peptide indicates the involvement of the hand of man. An engineered peptide is at least substantially free from at least one feature that is naturally associated or found in a naturally occurring peptide (e.g., length), and/or contains a modification (e.g., chemical modification or amino acid sequence) that is not present in the naturally occurring peptide. In some cases, engineered peptides of the present disclosure are selected through screening, rational design, and the like, to exhibit improved properties relative to naturally occurring peptides. For example, an engineered peptide of the present disclosure can have increased cellular toxicity relative to a naturally occurring peptide, and/or can have increased cell permeability relative to a naturally occurring peptide. In some cases, a peptide herein can have an N-terminal modification such as an acetyl modification. In some cases, a peptide herein can have a C-terminal modification such as an amide modification.

In some embodiments, an engineered peptide of the present disclosure has an half maximal inhibitory concentration (IC₅₀) of less than 200 μM, less than 165 μM, less than 100 μM, less than 95 μM, less than 90 μM, less than 85 μM, less than 80 μM, less than 75 μM, less than 70 μM, less than 65 μM, less than 60 μM, less than 55 μM, less than 50 μM, less than 45 μM, less than 40 μM, less than 35 μM, less than 30 μM, less than 25 μM, less than 20 μM, less than 15 μM, less than 10 μM, less than 5 μM, less than 1 μM, less than 100 nM, or less than 10 nM; as determined in an in vitro assay against a cancer cell line. In some embodiments, an engineered peptide of the present disclosure has an IC₅₀ of from about 1 nM to about 50 μM, 1 μM to about 50 μM, from about 100 μM to about 200 μM, from about 150 μM to about 200 μM, from about 10 μM to about 100 μM, from about 10 μM to about 60 μM, from about 10 μM to about 50 μM, from about 10 μM to about 45 μM, from about 5 μM to about 10 μM, from about 5 μM to about 15 μM, from about 40 μM to about 60 μM, from about 10 μM to about 30 μM, from about 10 μM to about 25 μM, from about 10 μM to about 20 μM, from about 10 μM to about 15 μM, from about 1 μM to about 5 μM, from about 500 nM to about 1 μM, from about 100 nM to about 500 nM, from about 10 nM to about 100 nM, from about 0.1 nM to about 1 nM, from about 0.01 nM to about 0.1 nM, or from about 1 nM to about 10 nM; as determined in an in vitro assay against a cancer cell line. In some embodiments, an engineered peptide of the present disclosure has an IC₅₀ of about 50 μM, about 49 μM, about 48 μM, about 47 μM, about 46 μM, about 45 μM, about 44 μM, about 43 μM, about 42 μM, about 41 μM, 40 μM, about 39 μM, about 38 μM, about 37 μM, about 36 μM, about 35 μM, about 34 μM, about 33 μM, about 32 μM, about 31 μM, about 30 μM, about 29 μM, about 28 μM, about 27 μM, about 26 μM, about 25 μM, about 24 μM, about 23 μM, about 22 μM, about 21 μM, about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, about 5 μM, about 4 μM, about 3 μM, about 2 μM, about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, about 9 nM, about 8 nM, about 7 nM, about 6 nM, about 5 nM, about 4 nM, about 3 nM, about 2 nM, about 1 nM, about 0.5 nM, or about 0.1 nM; as determined in an in vitro assay against a cancer cell line. In some embodiments, an engineered peptide of the present disclosure has an IC₅₀ of from 10 μM to 60 μM, from 10 μM to 50 μM, from 1 μM to 50 μM, from 10 μM to 45 μM, from 40 μM to 60 μM, from 5 μM to 10 μM, from 5 μM to 15 μM, from 10 μM to 30 μM, from 10 μM to 25 μM, from 10 μM to 15 μM, from 10 μM to 20 μM, from 1 μM to 5 μM, from 500 nM to 1 μM, from 500 nM to 800 nM, from 100 nM to 500 nM, from 50 nM to 250 nM, from 10 nM to 100 nM, from 0.1 nM to 1 nM, from 0.01 nM to 0.1 nM, or from 1 nM to 10 nM; as determined in an in vitro assay against a cancer cell line. In some embodiments, an engineered peptide of the present disclosure has an IC₅₀ of no more than 50 μM, no more than 49 μM, no more than 48 μM, no more than 47 μM, no more than 46 μM, no more than 45 μM, no more than 44 μM, no more than 43 μM, no more than 42 μM, no more than 41 μM, no more than 40 μM, no more than 39 μM, no more than 38 μM, no more than 37 μM, no more than 36 μM, no more than 35 μM, no more than 34 μM, no more than 33 μM, no more than 32 μM, no more than 31 μM, no more than 30 μM, no more than 29 μM, no more than 28 μM, no more than 27 μM, no more than 26 μM, no more than 25 μM, no more than 24 μM, no more than 23 μM, no more than 22 μM, no more than 21 μM, no more than 20 μM, no more than 19 μM, no more than 18 μM, no more than 17 μM, no more than 16 μM, no more than 15 μM, no more than 14 μM, no more than 13 μM, no more than 12 μM, no more than 11 μM, no more than 10 μM, no more than 9 μM, no more than 8 μM, no more than 7 μM, no more than 6 μM, no more than 5 μM, no more than 4 μM, no more than 3 μM, no more than 2 μM, no more than 1 μM, no more than 900 nM, no more than 800 nM, no more than 700 nM, no more than 600 nM, no more than 500 nM, no more than 400 nM, no more than 300 nM, no more than 200 nM, no more than 100 nM, no more than 90 nM, no more than 80 nM, no more than 70 nM, no more than 60 nM, no more than 50 nM, no more than 40 nM, no more than 30 nM, no more than 20 nM, no more than 10 nM, no more than 9 nM, no more than 8 nM, no more than 7 nM, no more than 6 nM, no more than 5 nM, no more than 4 nM, no more than 3 nM, no more than 2 nM, no more than 1 nM, no more than 0.5 nM, or no more than 0.1 nM; as determined in an in vitro assay against a cancer cell line.

In some embodiments, an engineered peptide of the present disclosure can comprise a portion of a naturally occurring peptide. For example, an engineered peptide of the present disclosure can comprise a portion of an alpha helical domain of a naturally occurring protein. As described herein, such portions can be further engineered in the development of the engineered peptides to deviate from the portion of the naturally occurring peptide. In some embodiments, an engineered peptide of the present disclosure can comprise, or can be derived from prior to engineering, a portion of an RB1 peptide, a CHEK2 peptide, an MDM2 peptide, a RAF1 peptide, a RAC1 peptide, an RRAS2 peptide, a FAC1 peptide, a PIK3Ca peptide, a PIK3R1 peptide, a VHL peptide, a DDX3X peptide, or a CTNNB1 peptide. In some embodiments, a therapeutic peptide of the present disclosure can have at least 80%, at least 85%, at least 90%, at least 95%, or at least 99% sequence identity to a contiguous portion of an RB1 peptide, a CHEK2 peptide, an MDM2 peptide, a RAF1 peptide, a RAC1 peptide, an RRAS2 peptide, a FAC1 peptide, a PIK3Ca peptide, a PIK3R1 peptide, a VHL peptide, a DDX3X peptide, or a CTNNB1 peptide.

In some cases, an engineered peptide can comprise, or be derived from a portion of a naturally occurring protein that is from about 5 amino acids to about 40 amino acids, from about 6 amino acids to about 40 amino acids, from about 7 amino acids to about 40 amino acids, from about 8 amino acids to about 40 amino acids, from about 9 amino acids to about 40 amino acids, from about 10 amino acids to about 40 amino acids, from about 11 amino acids to about 40 amino acids, from about 12 amino acids to about 40 amino acids, from about 13 amino acids to about 40 amino acids, from about 14 amino acids to about 40 amino acids, from about 15 amino acids to about 40 amino acids, from about 16 amino acids to about 40 amino acids, from about 17 amino acids to about 40 amino acids, from about 18 amino acids to about 40 amino acids, from about 19 amino acids to about 40 amino acids, from about 20 amino acids to about 40 amino acids, from about 21 amino acids to about 40 amino acids, from about 22 amino acids to about 40 amino acids, from about 23 amino acids to about 40 amino acids, from about 24 amino acids to about 40 amino acids, from about 25 amino acids to about 40 amino acids, from about 26 amino acids to about 40 amino acids, from about 27 amino acids to about 40 amino acids, from about 28 amino acids to about 40 amino acids, from about 29 amino acids to about 40 amino acids, from about 30 amino acids to about 40 amino acids, from about 31 amino acids to about 40 amino acids, from about 32 amino acids to about 40 amino acids, from about 33 amino acids to about 40 amino acids, from about 34 amino acids to about 40 amino acids, from about 35 amino acids to about 40 amino acids, from about 36 amino acids to about 40 amino acids, from about 37 amino acids to about 40 amino acids, from about 38 amino acids to about 40 amino acids, or from about 39 amino acids to about 40 amino acids. In some embodiments, an engineered peptide can comprise, or be derived from prior to engineered, a portion of a naturally occurring protein that is from about 5 amino acids to about 20 amino acids, from about 6 amino acids to about 20 amino acids, from about 7 amino acids to about 20 amino acids, from about 8 amino acids to about 20 amino acids, from about 9 amino acids to about 20 amino acids, from about 10 amino acids to about 20 amino acids, from about 11 amino acids to about 20 amino acids, from about 12 amino acids to about 20 amino acids, from about 13 amino acids to about 20 amino acids, from about 14 amino acids to about 20 amino acids, from about 15 amino acids to about 20 amino acids, from about 16 amino acids to about 20 amino acids, from about 17 amino acids to about 20 amino acids, from about 18 amino acids to about 20 amino acids, or from about 19 amino acids to about 20 amino acids. In some cases, an engineered peptide can comprise, or be derived from, a portion of a naturally occurring protein that is from 5 amino acids to 40 amino acids, from 6 amino acids to 40 amino acids, from 7 amino acids to 40 amino acids, from 8 amino acids to 40 amino acids, from 9 amino acids to 40 amino acids, from 10 amino acids to 40 amino acids, from 11 amino acids to 40 amino acids, from 12 amino acids to 40 amino acids, from 13 amino acids to 40 amino acids, from 14 amino acids to 40 amino acids, from 15 amino acids to 40 amino acids, from 16 amino acids to 40 amino acids, from 17 amino acids to 40 amino acids, from 18 amino acids to 40 amino acids, from 19 amino acids to 40 amino acids, from 20 amino acids to 40 amino acids, from 21 amino acids to 40 amino acids, from 22 amino acids to 40 amino acids, from 23 amino acids to 40 amino acids, from 24 amino acids to 40 amino acids, from 25 amino acids to 40 amino acids, from 26 amino acids to 40 amino acids, from 27 amino acids to 40 amino acids, from 28 amino acids to 40 amino acids, from 29 amino acids to 40 amino acids, from 30 amino acids to 40 amino acids, from 31 amino acids to 40 amino acids, from 32 amino acids to 40 amino acids, from 33 amino acids to 40 amino acids, from 34 amino acids to 40 amino acids, from 35 amino acids to 40 amino acids, from 36 amino acids to 40 amino acids, from 37 amino acids to 40 amino acids, from 38 amino acids to 40 amino acids, or from 39 amino acids to 40 amino acids. In some cases, an engineered peptide can comprise, or be derived from, a portion of a naturally occurring protein that is from 5 amino acids to 39 amino acids, from 6 amino acids to 39 amino acids, from 7 amino acids to 39 amino acids, from 8 amino acids to 39 amino acids, from 9 amino acids to 39 amino acids, from 10 amino acids to 39 amino acids, from 11 amino acids to 39 amino acids, from 12 amino acids to 39 amino acids, from 13 amino acids to 39 amino acids, from 14 amino acids to 39 amino acids, from 15 amino acids to 39 amino acids, from 16 amino acids to 39 amino acids, from 17 amino acids to 39 amino acids, from 18 amino acids to 39 amino acids, from 19 amino acids to 39 amino acids, from 20 amino acids to 39 amino acids, from 21 amino acids to 39 amino acids, from 22 amino acids to 39 amino acids, from 23 amino acids to 39 amino acids, from 24 amino acids to 39 amino acids, from 25 amino acids to 39 amino acids, from 26 amino acids to 39 amino acids, from 27 amino acids to 39 amino acids, from 28 amino acids to 39 amino acids, from 29 amino acids to 39 amino acids, from 30 amino acids to 39 amino acids, from 31 amino acids to 39 amino acids, from 32 amino acids to 39 amino acids, from 33 amino acids to 39 amino acids, from 34 amino acids to 39 amino acids, from 35 amino acids to 39 amino acids, from 36 amino acids to 39 amino acids, from 37 amino acids to 39 amino acids, or from 38 amino acids to 39 amino acids. In some embodiments, an engineered peptide can comprise, or be derived from, a portion of a naturally occurring protein that is from 5 amino acids to 20 amino acids, from 6 amino acids to 20 amino acids, from 7 amino acids to 20 amino acids, from 8 amino acids to 20 amino acids, from 9 amino acids to 20 amino acids, from 10 amino acids to 20 amino acids, from 10 amino acids to 15 amino acids, from 11 amino acids to 20 amino acids, from 12 amino acids to 20 amino acids, from 13 amino acids to 20 amino acids, from 14 amino acids to 20 amino acids, from 15 amino acids to 20 amino acids, from 16 amino acids to 20 amino acids, from 17 amino acids to 20 amino acids, from 18 amino acids to 20 amino acids, or from 19 amino acids to 20 amino acids. In some embodiments, an engineered peptide can comprise, or be derived from, a portion of a naturally occurring protein that is at most 40 amino acids, at most 39 amino acids, at most 38 amino acids, at most 37 amino acids, at most 36 amino acids, at most 35 amino acids, at most 34 amino acids, at most 33 amino acids, at most 32 amino acids, at most 31 amino acids, at most 30 amino acids, at most 29 amino acids, at most 28 amino acids, at most 27 amino acids, at most 26 amino acids, at most 25 amino acids, at most 24 amino acids, at most 23 amino acids, at most 22 amino acids, at most 21 amino acids, at most 20 amino acids, at most 19 amino acids, at most 18 amino acids, at most 17 amino acids, at most 16 amino acids, at most 15 amino acids, at most 14 amino acids, at most 13 amino acids, at most 12 amino acids, at most 11 amino acids, at most 10 amino acids, at most 9 amino acids, at most 8 amino acids, at most 7 amino acids, at most 6 amino acids, or at most 5 amino acids. In some embodiments, an engineered peptide can comprise, or be derived from, a portion of a naturally occurring protein that is 40 amino acids, 39 amino acids, 38 amino acids, 37 amino acids, 36 amino acids, 35 amino acids, 34 amino acids, 33 amino acids, 32 amino acids, 31 amino acids, 30 amino acids, 29 amino acids, 28 amino acids, 27 amino acids, 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, or 5 amino acids.

In some cases, an engineered peptide can consist of a portion of a naturally occurring protein or of a portion derived a naturally occurring protein that is from about 5 amino acids to about 40 amino acids, from about 6 amino acids to about 40 amino acids, from about 7 amino acids to about 40 amino acids, from about 8 amino acids to about 40 amino acids, from about 9 amino acids to about 40 amino acids, from about 10 amino acids to about 40 amino acids, from about 11 amino acids to about 40 amino acids, from about 12 amino acids to about 40 amino acids, from about 13 amino acids to about 40 amino acids, from about 14 amino acids to about 40 amino acids, from about 15 amino acids to about 40 amino acids, from about 16 amino acids to about 40 amino acids, from about 17 amino acids to about 40 amino acids, from about 18 amino acids to about 40 amino acids, from about 19 amino acids to about 40 amino acids, from about 20 amino acids to about 40 amino acids, from about 21 amino acids to about 40 amino acids, from about 22 amino acids to about 40 amino acids, from about 23 amino acids to about 40 amino acids, from about 24 amino acids to about 40 amino acids, from about 25 amino acids to about 40 amino acids, from about 26 amino acids to about 40 amino acids, from about 27 amino acids to about 40 amino acids, from about 28 amino acids to about 40 amino acids, from about 29 amino acids to about 40 amino acids, from about 30 amino acids to about 40 amino acids, from about 31 amino acids to about 40 amino acids, from about 32 amino acids to about 40 amino acids, from about 33 amino acids to about 40 amino acids, from about 34 amino acids to about 40 amino acids, from about 35 amino acids to about 40 amino acids, from about 36 amino acids to about 40 amino acids, from about 37 amino acids to about 40 amino acids, from about 38 amino acids to about 40 amino acids, or from about 39 amino acids to about 40 amino acids. In some embodiments, an engineered peptide can consist of a portion of a naturally occurring protein or of a portion derived a naturally occurring protein that is from about 5 amino acids to about 20 amino acids, from about 6 amino acids to about 20 amino acids, from about 7 amino acids to about 20 amino acids, from about 8 amino acids to about 20 amino acids, from about 9 amino acids to about 20 amino acids, from about 10 amino acids to about 20 amino acids, from about 11 amino acids to about 20 amino acids, from about 12 amino acids to about 20 amino acids, from about 13 amino acids to about 20 amino acids, from about 14 amino acids to about 20 amino acids, from about 15 amino acids to about 20 amino acids, from about 16 amino acids to about 20 amino acids, from about 17 amino acids to about 20 amino acids, from about 18 amino acids to about 20 amino acids, or from about 19 amino acids to about 20 amino acids. In some cases, an engineered peptide can consist of a portion of a naturally occurring protein or of a portion derived a naturally occurring protein that is from 5 amino acids to 40 amino acids, from 6 amino acids to 40 amino acids, from 7 amino acids to 40 amino acids, from 8 amino acids to 40 amino acids, from 9 amino acids to 40 amino acids, from 10 amino acids to 40 amino acids, from 11 amino acids to 40 amino acids, from 12 amino acids to 40 amino acids, from 13 amino acids to 40 amino acids, from 14 amino acids to 40 amino acids, from 15 amino acids to 40 amino acids, from 16 amino acids to 40 amino acids, from 17 amino acids to 40 amino acids, from 18 amino acids to 40 amino acids, from 19 amino acids to 40 amino acids, from 20 amino acids to 40 amino acids, from 21 amino acids to 40 amino acids, from 22 amino acids to 40 amino acids, from 23 amino acids to 40 amino acids, from 24 amino acids to 40 amino acids, from 25 amino acids to 40 amino acids, from 26 amino acids to 40 amino acids, from 27 amino acids to 40 amino acids, from 28 amino acids to 40 amino acids, from 29 amino acids to 40 amino acids, from 30 amino acids to 40 amino acids, from 31 amino acids to 40 amino acids, from 32 amino acids to 40 amino acids, from 33 amino acids to 40 amino acids, from 34 amino acids to 40 amino acids, from 35 amino acids to 40 amino acids, from 36 amino acids to 40 amino acids, from 37 amino acids to 40 amino acids, from 38 amino acids to 40 amino acids, or from 39 amino acids to 40 amino acids. In some cases, an engineered peptide can consist of a portion of a naturally occurring protein or of a portion derived a naturally occurring protein that is from 5 amino acids to 39 amino acids, from 6 amino acids to 39 amino acids, from 7 amino acids to 39 amino acids, from 8 amino acids to 39 amino acids, from 9 amino acids to 39 amino acids, from 10 amino acids to 39 amino acids, from 11 amino acids to 39 amino acids, from 12 amino acids to 39 amino acids, from 13 amino acids to 39 amino acids, from 14 amino acids to 39 amino acids, from 15 amino acids to 39 amino acids, from 16 amino acids to 39 amino acids, from 17 amino acids to 39 amino acids, from 18 amino acids to 39 amino acids, from 19 amino acids to 39 amino acids, from 20 amino acids to 39 amino acids, from 21 amino acids to 39 amino acids, from 22 amino acids to 39 amino acids, from 23 amino acids to 39 amino acids, from 24 amino acids to 39 amino acids, from 25 amino acids to 39 amino acids, from 26 amino acids to 39 amino acids, from 27 amino acids to 39 amino acids, from 28 amino acids to 39 amino acids, from 29 amino acids to 39 amino acids, from 30 amino acids to 39 amino acids, from 31 amino acids to 39 amino acids, from 32 amino acids to 39 amino acids, from 33 amino acids to 39 amino acids, from 34 amino acids to 39 amino acids, from 35 amino acids to 39 amino acids, from 36 amino acids to 39 amino acids, from 37 amino acids to 39 amino acids, or from 38 amino acids to 39 amino acids. In some embodiments, an engineered peptide can consist of a portion of a naturally occurring protein or of a portion derived a naturally occurring protein that is from 5 amino acids to 20 amino acids, from 6 amino acids to 20 amino acids, from 7 amino acids to 20 amino acids, from 8 amino acids to 20 amino acids, from 9 amino acids to 20 amino acids, from 10 amino acids to 20 amino acids, from 10 amino acids to 15 amino acids, from 11 amino acids to 20 amino acids, from 12 amino acids to 20 amino acids, from 13 amino acids to 20 amino acids, from 14 amino acids to 20 amino acids, from 15 amino acids to 20 amino acids, from 16 amino acids to 20 amino acids, from 17 amino acids to 20 amino acids, from 18 amino acids to 20 amino acids, or from 19 amino acids to 20 amino acids. In some embodiments, an engineered peptide can consist of a portion of a naturally occurring protein or of a portion derived a naturally occurring protein that is at most 40 amino acids, at most 39 amino acids, at most 38 amino acids, at most 37 amino acids, at most 36 amino acids, at most 35 amino acids, at most 34 amino acids, at most 33 amino acids, at most 32 amino acids, at most 31 amino acids, at most 30 amino acids, at most 29 amino acids, at most 28 amino acids, at most 27 amino acids, at most 26 amino acids, at most 25 amino acids, at most 24 amino acids, at most 23 amino acids, at most 22 amino acids, at most 21 amino acids, at most 20 amino acids, at most 19 amino acids, at most 18 amino acids, at most 17 amino acids, at most 16 amino acids, at most 15 amino acids, at most 14 amino acids, at most 13 amino acids, at most 12 amino acids, at most 11 amino acids, at most 10 amino acids, at most 9 amino acids, at most 8 amino acids, at most 7 amino acids, at most 6 amino acids, or at most 5 amino acids. In some embodiments, an engineered peptide can consist of a portion of a naturally occurring protein or of a portion derived a naturally occurring protein that is 40 amino acids, 39 amino acids, 38 amino acids, 37 amino acids, 36 amino acids, 35 amino acids, 34 amino acids, 33 amino acids, 32 amino acids, 31 amino acids, 30 amino acids, 29 amino acids, 28 amino acids, 27 amino acids, 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, or 5 amino acids in length.

An engineered peptide of the present disclosure can have varying lengths depending on their functionality. In some cases, engineered peptides of the present disclosure are short peptides of low molecular weight. In some embodiments, an engineered peptide of the present disclosure can have a length of at most 40 amino acids, at most 39 amino acids, at most 38 amino acids, at most 37 amino acids, at most 36 amino acids, at most 35 amino acids, at most 34 amino acids, at most 33 amino acids, at most 32 amino acids, at most 31 amino acids, at most 30 amino acids, at most 29 amino acids, at most 28 amino acids, at most 27 amino acids, at most 26 amino acids, at most 25 amino acids, at most 24 amino acids, at most 23 amino acids, at most 22 amino acids, at most 21 amino acids, at most 20 amino acids, at most 19 amino acids, at most 18 amino acids, at most 17 amino acids, at most 16 amino acids, at most 15 amino acids, at most 14 amino acids, at most 13 amino acids, at most 12 amino acids, at most 11 amino acids, at most 10 amino acids, at most 9 amino acids, at most 8 amino acids, at most 7 amino acids, at most 6 amino acids, or at most 5 amino acids. In some embodiments, an engineered peptide of the present disclosure can have a length of from about 5 amino acids to about 40 amino acids, from about 6 amino acids to about 40 amino acids, from about 7 amino acids to about 40 amino acids, from about 8 amino acids to about 40 amino acids, from about 9 amino acids to about 40 amino acids, from about 10 amino acids to about 40 amino acids, from about 11 amino acids to about 40 amino acids, from about 12 amino acids to about 40 amino acids, from about 13 amino acids to about 40 amino acids, from about 14 amino acids to about 40 amino acids, from about 15 amino acids to about 40 amino acids, from about 16 amino acids to about 40 amino acids, from about 17 amino acids to about 40 amino acids, from about 18 amino acids to about 40 amino acids, from about 19 amino acids to about 40 amino acids, from about 20 amino acids to about 40 amino acids, from about 21 amino acids to about 40 amino acids, from about 22 amino acids to about 40 amino acids, from about 23 amino acids to about 40 amino acids, from about 24 amino acids to about 40 amino acids, from about 25 amino acids to about 40 amino acids, from about 26 amino acids to about 40 amino acids, from about 27 amino acids to about 40 amino acids, from about 28 amino acids to about 40 amino acids, from about 29 amino acids to about 40 amino acids, from about 30 amino acids to about 40 amino acids, from about 31 amino acids to about 40 amino acids, from about 32 amino acids to about 40 amino acids, from about 33 amino acids to about 40 amino acids, from about 34 amino acids to about 40 amino acids, from about 35 amino acids to about 40 amino acids, from about 36 amino acids to about 40 amino acids, from about 37 amino acids to about 40 amino acids, from about 38 amino acids to about 40 amino acids, or from about 39 amino acids to about 40 amino acids. In some embodiments, an engineered peptide of the present disclosure can have a length of from about 5 amino acids to about 20 amino acids, from about 6 amino acids to about 20 amino acids, from about 7 amino acids to about 20 amino acids, from about 8 amino acids to about 20 amino acids, from about 9 amino acids to about 20 amino acids, from about 10 amino acids to about 20 amino acids, from about 10 amino acids to about 15 amino acids, from about 11 amino acids to about 20 amino acids, from about 12 amino acids to about 20 amino acids, from about 13 amino acids to about 20 amino acids, from about 14 amino acids to about 20 amino acids, from about 15 amino acids to about 20 amino acids, from about 16 amino acids to about 20 amino acids, from about 17 amino acids to about 20 amino acids, from about 18 amino acids to about 20 amino acids, or from about 19 amino acids to about 20 amino acids. In some cases, an engineered peptide of the present disclosure can have a length of from 5 amino acids to 40 amino acids, from 6 amino acids to 40 amino acids, from 7 amino acids to 40 amino acids, from 8 amino acids to 40 amino acids, from 9 amino acids to 40 amino acids, from 10 amino acids to 40 amino acids, from 11 amino acids to 40 amino acids, from 12 amino acids to 40 amino acids, from 13 amino acids to 40 amino acids, from 14 amino acids to 40 amino acids, from 15 amino acids to 40 amino acids, from 16 amino acids to 40 amino acids, from 17 amino acids to 40 amino acids, from 18 amino acids to 40 amino acids, from 19 amino acids to 40 amino acids, from 20 amino acids to 40 amino acids, from 21 amino acids to 40 amino acids, from 22 amino acids to 40 amino acids, from 23 amino acids to 40 amino acids, from 24 amino acids to 40 amino acids, from 25 amino acids to 40 amino acids, from 26 amino acids to 40 amino acids, from 27 amino acids to 40 amino acids, from 28 amino acids to 40 amino acids, from 29 amino acids to 40 amino acids, from 30 amino acids to 40 amino acids, from 31 amino acids to 40 amino acids, from 32 amino acids to 40 amino acids, from 33 amino acids to 40 amino acids, from 34 amino acids to 40 amino acids, from 35 amino acids to 40 amino acids, from 36 amino acids to 40 amino acids, from 37 amino acids to 40 amino acids, from 38 amino acids to 40 amino acids, or from 39 amino acids to 40 amino acids. In some cases, an engineered peptide can have a length of from 5 amino acids to 39 amino acids, from 6 amino acids to 39 amino acids, from 7 amino acids to 39 amino acids, from 8 amino acids to 39 amino acids, from 9 amino acids to 39 amino acids, from 10 amino acids to 39 amino acids, from 11 amino acids to 39 amino acids, from 12 amino acids to 39 amino acids, from 13 amino acids to 39 amino acids, from 14 amino acids to 39 amino acids, from 15 amino acids to 39 amino acids, from 16 amino acids to 39 amino acids, from 17 amino acids to 39 amino acids, from 18 amino acids to 39 amino acids, from 19 amino acids to 39 amino acids, from 20 amino acids to 39 amino acids, from 21 amino acids to 39 amino acids, from 22 amino acids to 39 amino acids, from 23 amino acids to 39 amino acids, from 24 amino acids to 39 amino acids, from 25 amino acids to 39 amino acids, from 26 amino acids to 39 amino acids, from 27 amino acids to 39 amino acids, from 28 amino acids to 39 amino acids, from 29 amino acids to 39 amino acids, from 30 amino acids to 39 amino acids, from 31 amino acids to 39 amino acids, from 32 amino acids to 39 amino acids, from 33 amino acids to 39 amino acids, from 34 amino acids to 39 amino acids, from 35 amino acids to 39 amino acids, from 36 amino acids to 39 amino acids, from 37 amino acids to 39 amino acids, or from 38 amino acids to 39 amino acids. In some cases, an engineered peptide of the present disclosure can have a length of from 5 amino acids to 20 amino acids, from 6 amino acids to 20 amino acids, from 7 amino acids to 20 amino acids, from 8 amino acids to 20 amino acids, from 9 amino acids to 20 amino acids, from 10 amino acids to 20 amino acids, from 11 amino acids to 20 amino acids, from 12 amino acids to 20 amino acids, from 13 amino acids to 20 amino acids, from 14 amino acids to 20 amino acids, from 15 amino acids to 20 amino acids, from 16 amino acids to 20 amino acids, from 17 amino acids to 20 amino acids, from 18 amino acids to 20 amino acids, or from 19 amino acids to 20 amino acids. In some embodiments, an engineered peptide can have a length of 40 amino acids, 39 amino acids, 38 amino acids, 37 amino acids, 36 amino acids, 35 amino acids, 34 amino acids, 33 amino acids, 32 amino acids, 31 amino acids, 30 amino acids, 29 amino acids, 28 amino acids, 27 amino acids, 26 amino acids, 25 amino acids, 24 amino acids, 23 amino acids, 22 amino acids, 21 amino acids, 20 amino acids, 19 amino acids, 18 amino acids, 17 amino acids, 16 amino acids, 15 amino acids, 14 amino acids, 13 amino acids, 12 amino acids, 11 amino acids, 10 amino acids, 9 amino acids, 8 amino acids, 7 amino acids, 6 amino acids, or 5 amino acids. In some embodiments, an engineered peptide can have a length of 10 amino acids. In some embodiments, an engineered peptide can have a length of 11 amino acids. In some embodiments, an engineered peptide can have a length of 12 amino acids. In some embodiments, an engineered peptide can have a length of 13 amino acids. In some embodiments, an engineered peptide can have a length of 14 amino acids. In some embodiments, an engineered peptide can have a length of 15 amino acids.

In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1. In some embodiments, an engineered peptide can have a polypeptide sequence of SEQ ID NO: 1. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2. In some embodiments, an engineered peptide can have a polypeptide sequence of SEQ ID NO: 2. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3. In some embodiments, an engineered peptide can have a polypeptide sequence of SEQ ID NO: 3.

In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1 and further comprise a peptide staple. In some embodiments, an engineered peptide can have a polypeptide sequence of SEQ ID NO: 1 and further comprise a peptide staple. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2 and further comprise a peptide staple. In some embodiments, an engineered peptide can have a polypeptide sequence of SEQ ID NO: 2 and further comprise a peptide staple. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3 and further comprise a peptide staple. In some embodiments, an engineered peptide can have a polypeptide sequence of SEQ ID NO: 3 and further comprise a peptide staple. The peptide staple can be (i, i+n) as previously described. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1 and further comprise a peptide staple (i, i+7), wherein i is 3, the (i) residue is R8 and the (i+7) residue is S5. In some embodiments, an engineered peptide can have a polypeptide sequence of SEQ ID NO: 1 and further comprise a peptide staple further comprise a peptide staple (i, i+7), wherein i is 3, the (i) residue is R8 and the (i+7) residue is S5. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 2 and further comprise a peptide staple (i, i+7), wherein i is 3, the (i) residue is R8 and the (i+7) residue is S5. In some embodiments, an engineered peptide can have a polypeptide sequence of SEQ ID NO: 2 and further comprise a peptide staple (i, i+7), wherein i is 3, the (i) residue is R8 and the (i+7) residue is S5. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 3 and further comprise a peptide staple (i, i+7), wherein i is 4, the (i) residue is R8 and the (i+7) residue is S5. In some embodiments, an engineered peptide can have a polypeptide sequence of SEQ ID NO: 3 and further comprise a peptide staple (i, i+7), wherein i is 4, the (i) residue is R8 and the (i+7) residue is S5.

In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 4. In some embodiments, an engineered peptide can have a polypeptide sequence of SEQ ID NO: 4. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5. In some embodiments, an engineered peptide can have a polypeptide sequence of SEQ ID NO: 5. In some embodiments, an engineered peptide can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6. In some embodiments, an engineered peptide can have a polypeptide sequence of SEQ ID NO: 6. In some embodiments, an engineered peptide can be a stapled peptide having a polypeptide sequence of SEQ ID NO: 4, SEQ ID NO: 5, or SEQ ID NO: 6.

### Pharmaceutical Compositions

Engineered peptides as described herein can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some embodiments, the peptides disclosed herein can be formulated as a salt of the peptide. In some cases, the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof. In some cases, a salt comprises an acetate salt. In some cases, a salt comprises a trifluoroacetate (TFA) salt.

In some aspects, pharmaceutically acceptable salts include, but are not limited to, metal salts such as a sodium salt, a potassium salt, a cesium salt and the like; an alkaline earth metal salt such as a calcium salt, a magnesium salt and the like; an organic amine salts such as a triethylamine salt, a pyridine salt, a picoline salt, a ethanolamine salt, a triethanolamine salt, a dicyclohexylamine salt, a N,N′-dibenzylethylenediamine salt and the like; an inorganic acid salt such as a hydrochloride, a hydrobromide, a phosphate, a sulphate and the like; an organic acid salt such as a citrate, a lactate, a tartrate, a maleate, a fumarate, a mandelate, an acetate, a dichloroacetate, a trifluoroacetate, an oxalate, a formate and the like; a sulfonate such as a methanesulfonate, a benzenesulfonate, a p-toluenesulfonate and the like; and an amino acid salt such as an arginate, an asparginate, a glutamate and the like. In some cases, a pharmaceutically acceptable salt comprises an acetate salt.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a trigylceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

Also disclosed herein are kits comprising the pharmaceutical compositions herein. In some cases, a kit comprises a container. A container can be any material such as a glass, a metal, a plastic or a combination thereof. A kit can comprise an engineered peptide disclosed herein such as a peptide of SEQ ID NOs: 1-6 and optionally, a pharmaceutically acceptable: excipient, carrier, or diluent.

### Methods of Screening

Also disclosed herein are methods of screening for engineered peptides of the present disclosure. In some embodiments, an initial screen can be utilized to select for portions of polypeptides that exhibit a property of interest. An example of such screening can be found in WO2021041953, which is incorporated by reference in its entirety. In some embodiments, an initial screen can be used to tile portions of oncogenes that display cell toxicity. Hits from the initial screen can be further engineered as described herein to produce improved engineered peptides with enhanced efficacy relative to naturally occurring portions of oncogenes.

In some embodiments, peptide candidates found in an initial screen to display cellular toxicity are curated to select for certain properties using external databases such as Unitprot, Genebank, and PDB. For example, in some instances peptide candidates can be selected based on their propensity to form an alpha helical structure in the context of a full length peptide. As described herein, and without wishing to be bound by theory, an alpha-helical engineered peptide of the present disclosure can have improved cell permeability, relative to a corresponding non-alpha helical peptide. Alpha helical propensity can be determined by comparing the peptide candidates to deposited protein structures showing the presence of the peptide candidate in an annotated alpha helical structure in the deposited protein structure.

Curated peptide candidates (e.g. peptide candidates selected for cellular toxicity in an initial screen and for their propensity to form alpha helices based on deposited structures) can be further engineered to improve their activity. In some instances, mutagenesis can be utilized to improve the activity of the candidate peptides. In some instances, high throughput screening can be utilized to screen libraries of rationally designed engineered peptides based on peptide candidates selected in the initial screen. In some instances, computational screening (e.g., computational alanine scanning) can be performed to map key residues to improve the properties of the peptide candidate. In some cases, both methods can be combined to generate engineered peptides with improved properties relative to the peptide candidates.

Advanced candidates selected by screening with improved properties relative to the peptide candidates can be further optimized to improve efficacy. For example, each advanced candidate can be modeled to predict the key interactions between the candidate peptide and the target. Based on the modeling, the length of the candidate can be modified to include key residues. In some embodiments, based on the modeling, the length of the candidate can be modified to minimize the length of the peptide while still including key residues. Further, modifications such as stapling can be employed as described herein to stabilize the secondary structure (e.g. alpha helical structure) of the therapeutic peptide by reducing the entropic penalty for adopting the structure.

### Methods of Treatment

Engineered peptides as described herein can be used to treat diseases or conditions upon administration to a subject in need thereof. Without wishing to be bound by theory, engineered peptides of the present disclosure exhibit cellular toxicity that allows for treatment of diseases such as cancer. In some instances, contacting an engineered peptide of the present disclosure with a cancer cell can arrest cancer cell growth. In some instances, contacting an engineered peptide of the present disclosure with a cancer cell can inhibit cancer cell proliferation. In some instances, contacting an engineered peptide of the present disclosure can reduce the size of a cancer cell (e.g. can decrease tumor volume). In some instances, contacting an engineered peptide of the present disclosure can induce apoptosis or cell death of the cancer cell. In some cases, the arrest of cancer cell growth, the inhibition of cancer cell proliferation, the reduction of the tumor volume, or the induction of apoptosis of a cell by a peptide of the present disclosure can be compared to the results from a control peptide.

An engineered peptide of the present disclosure can be used to treat cancer upon administration. In some embodiments, the cancer is selected from the group consisting of: adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In some embodiments, the cancer is a colorectal cancer. In some embodiments, the cancer is a lung cancer. In some embodiments, the cancer is a melanoma cancer. In some embodiments, the cancer is a breast cancer. In some embodiments, the cancer is a pancreatic cancer, a bladder cancer, a triple negative breast cancer, an ovarian cancer, a juvenile myelomonocytic leukemia (JMML), a hematologic malignancy, a cholangiocarcinoma, a uterine cancer, a cervical cancer, a testicular cancer, or any combination thereof. In some embodiments, the cancer is a cancer comprising a KRAS mutation. In some cases, a KRAS mutation can comprise a G12V mutation, a G12C mutation, or a G12D mutation of KRAS. In some cases, a KRAS mutation can comprise a G13D mutation, a G12R mutation, or a G12A mutation of KRAS. For example, an administration of a peptide herein can be used to treat any cancer with a KRAS mutation, such as those with a G12V mutation, a G12C mutation, or a G12D mutation.

Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, intratumoral injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

Dosing may include single or multiple administrations of a pharmaceutical composition described herein. Examples include: multiple times a day, daily, every other day, 1, 2, 3, 5, 6, or 7 times a week, weekly, or less often, a single administration, a course of treatment involving several treatments on a regular or irregular basis, or multiple administrations for a period of time until the treatment of a cancer is achieved. In some embodiments, dosing may be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years. In some cases, dosing can occur every day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 1 year, 2 years, or as needed. The dosing regimen, including the regularity of and mode of administration, may be dependent on factors including but not limited to the subject being treated; the severity of the condition; the manner of administration, or the presence of one or more additional diseases or conditions.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, from about 1 mg/kg to about 25 mg/kg, from about 1 mg/kg to about 300 mg/kg, from about 0.001 mg/kg to about 300 mg/kg, from about 10 mg/kg to about 300 mg/kg, from about 50 mg/kg to about 300 mg/kg, from about 75 mg/kg to about 300 mg/kg, from about 100 mg/kg to about 300 mg/kg, from about 200 mg/kg to about 300 mg/kg, from about 5 mg/kg to about 250 mg/kg, from about 50 mg/kg to about 250 mg/kg, from about 100 mg/kg to about 250 mg/kg, from about 150 mg/kg to about 250 mg/kg, from about 50 mg/kg to about 150 mg/kg, or from about 100 mg/kg to about 200 mg/kg of subject body weight per day, one or more times a day, to obtain the desired therapeutic, diagnostic, or prophylactic, effect. In some examples, a pharmaceutical composition disclosed herein can be administered at dosage levels sufficient to deliver from 0.0001 mg/kg to 100 mg/kg, from 0.001 mg/kg to 0.05 mg/kg, from 0.005 mg/kg to 0.05 mg/kg, from 0.001 mg/kg to 0.005 mg/kg, from 0.05 mg/kg to 0.5 mg/kg, from 0.01 mg/kg to 50 mg/kg, from 0.1 mg/kg to 40 mg/kg, from 0.5 mg/kg to 30 mg/kg, from 0.01 mg/kg to 10 mg/kg, from 0.1 mg/kg to 10 mg/kg, from 5 mg/kg to 20 mg/kg, from 5 mg/kg to 15 mg/kg, from 8 mg/kg to 12 mg/kg, from 5 mg/kg to 10 mg/kg, from 40 mg/kg to 70 mg/kg, from 50 mg/kg to 70 mg/kg, from 55 mg/kg to 65 mg/kg, from 58 mg/kg to 64 mg/kg, from 59 mg/kg to 61 mg/kg, from 1 mg/kg to 25 mg/kg, from 1 mg/kg to 300 mg/kg, from 0.001 mg/kg to 300 mg/kg, from 10 mg/kg to 300 mg/kg, from 50 mg/kg to 300 mg/kg, from 75 mg/kg to 300 mg/kg, from 100 mg/kg to 300 mg/kg, from 200 mg/kg to 300 mg/kg, from 5 mg/kg to 250 mg/kg, from 50 mg/kg to 250 mg/kg, from 100 mg/kg to 250 mg/kg, from 150 mg/kg to 250 mg/kg, from 50 mg/kg to 150 mg/kg, or from 100 mg/kg to 200 mg/kg subject body weight per day, one or more times a day, to obtain the desired therapeutic, diagnostic, or prophylactic, effect. In some embodiments, a pharmaceutical composition disclosed herein can be administered at dosage levels sufficient to deliver about 0.0001 mg/kg, about 0.001 mg/kg, about 0.005 mg/kg, about 0.01 mg/kg, about 0.05 mg/kg, about 0.1 mg/kg, about 0.5 mg/kg, about 1 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, or about 300 mg/kg, of subject body weight per day, one or more times a day, to obtain the desired therapeutic, diagnostic, or prophylactic effect. In some embodiments, a pharmaceutical composition disclosed herein can be administered at dosage levels sufficient to deliver 0.0001 mg/kg, 0.001 mg/kg, 0.005 mg/kg, 0.01 mg/kg, 0.05 mg/kg, 0.1 mg/kg, 0.5 mg/kg, 1 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 20 mg/kg, 25 mg/kg, 30 mg/kg, 35 mg/kg, 40 mg/kg, 45 mg/kg, 50 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 70 mg/kg, 75 mg/kg, 80 mg/kg, 85 mg/kg, 90 mg/kg, 95 mg/kg, 100 mg/kg, 120 mg/kg, 140 mg/kg, 160 mg/kg, 180 mg/kg, 200 mg/kg, 220 mg/kg, 240 mg/kg, 260 mg/kg, 280 mg/kg, or 300 mg/kg, of subject body weight per day, one or more times a day, to obtain the desired therapeutic, diagnostic, or prophylactic, effect. In some embodiments, the dosage levels of the pharmaceutical composition as disclosed herein are administered to a mammal, including, but not limited to a human, a non-human primate, a dog, a cat, a hamster, a guinea pig, a rat, or a mouse. In some embodiments a subject in need thereof is a mammal, including, but not limited to a human, a non-human primate, a dog, a cat, a hamster, a guinea pig, a rat, or a mouse. In some embodiments, the subject is an infant. The infant can be up to 24 months old. In some embodiments, the subject is a child. The child may be 2 years to 21 years old. In some embodiments, the subject is an adult. Adults may be 21 years old or more. In some embodiments, the adult is of advanced age, such as 65 years or older.

In some embodiments, a method can comprise administering a second therapy. In some cases, a second therapy can be administered concurrently or consecutively with a composition disclosed herein. In some cases, a second therapy can comprise a chemotherapy, a radiation therapy, a hormone therapy, a hyperthermia therapy, an immunotherapy, a photodynamic therapy, a stem cell therapy, a surgery, a targeted therapy, or any combination thereof. In some cases, a chemotherapy can comprise an altretamine, a bendamustine, a busulfan, a carboplatin, a carmustine, a chlorambucil, a cisplatin, a cyclophosphamide, a dacarbazine, an ifosfamide, a lomustine, a mechlorethamine, a melphalan, an oxaliplatin, a temozolomide, a thiotepa, a trabectedin, or any combination thereof. In some cases, a chemotherapy can comprise an altretamine, a bendamustine, a busulfan, a carboplatin, a carmustine, a chlorambucil, a cisplatin, a cyclophosphamide, a dacarbazine, an ifosfamide, a lomustine, a mechlorethamine, a melphalan, an oxaliplatin, a temozolomide, a thiotepa, a trabectedin, a salt of any of these, or any combination thereof. In some cases, a chemotherapy can comprise a carmustine, a lomustine, a streptozocin, an azacitidine, a 5-fluorouracil (5-fu), a 6-mercaptopurine (6-mp), a capecitabine, a cladribine, a clofarabine, a cytarabine (ara-c), a decitabine, a floxuridine, a fludarabine, a gemcitabine, a hydroxyurea, a methotrexate, a nelarabine, a pemetrexed, a pentostatin, a pralatrexate, a thioguanine, a trifluridine/tipiracil combination, a daunorubicin, a doxorubicin, an epirubicin, an idarubicin, a valrubicin, a irinotecan, a topotecan, an etoposide (vp-16), a mitoxantrone, a teniposide, a vinblastine, a vincristine, a vinorelbine, a vinorelbine, a prednisone, a methylprednisolone, a dexamethasone, a all-trans-retinoic acid, an arsenic trioxide, an asparaginase, a eribulin, a hydroxyurea, a ixabepilone, a mitotane, an omacetaxine, a pegaspargase, a procarbazine, a romidepsin, a vorinostat, a salt of any of these, or any combination thereof. In some cases, a radiation therapy can comprise an internal radiation therapy or an external radiation therapy. In some cases, a hormone therapy can comprise an anastrozole, an exemestane, a letrozole, a tamoxifen, a raloxifene, a fulvestrant, a toremifene, a goserelin, a leuprolide, a triptorelinan apalutamide, an enzalutamide, a darolutamide, a bicalutamide, a flutamide, a nilutamide, an abiraterone, a ketoconazole, a degarelix, a medroxyprogesterone, a megestrol, an etrozole, an anastrozole, and an exemestane, a mitotane, a fulvestrant, a toremifene, a salt of any of these, or any combination thereof. In some cases, an immunotherapy can comprise a checkpoint inhibitor, an atezolizumab, an avelumab, a dostarlizumab, a durvalumab, an ipilimumab, an nivolumab, an pembrolizumab, an interferon, an interleukin, a Bacillus Calmette-Guerin (BCG), a oncolytic virus therapy, a CAR T-Cell therapy, a CAR cell therapy, a cancer vaccine, a salt of any of these, or any combination thereof. In some cases, a targeted therapy can comprise an angiogenesis inhibitor, a monoclonal antibody, a proteasome inhibitor, a signal transduction inhibitor. In some cases, a targeted therapy can comprise a denosumab, a romidepsin, a ofatumumab, a pazopanib, an everolimus, a nilotinib, a temsirolimus, a lapatinib, a sunitinib malate, a dasatinib, a vorinostat, a panitumumab, a sorafenib tosylate, a erlotinib, a bevacizumab, a cetuximab, a tositumomab, a bortezomib, a gefitinib, an ibritumomab tiuxetan, an alemtuzumab, an imatinib, a gemtuzumab ozogamicin, a denileukin diftitox, a trastuzumab, a rituximab, a salt of any of these, or any combination thereof.

In some embodiments, a method can comprise diagnosing a patient with a cancer prior to, during, or after treatment with a composition herein. In some cases, the diagnosing can comprise a physical examination, a blood test, an imaging assay, a neurological examination, an immunological assay, or any combination thereof. In some cases, an imaging assay can comprise an X-ray, a computed tomography (CT scan), a magnetic resonance imaging (MRI), an ultrasound, a positron emission tomography (PET scan), a fluoroscopy, or any combination thereof.

### EXAMPLES

For a better understanding of the present disclosure and of its many advantages, the following examples are given by way of illustration and without limiting the scope of this disclosure.

### Example 1: Initial Screening of Therapeutic Peptides Against Cancer

This example demonstrates initial screening of 40 mer peptides generated from various oncogenes. Each peptide was screened for its ability to exhibit cell toxicity, and variants displaying cell toxicity were utilized in the subsequent examples.

### Design of Peptide Coding Gene Fragment Libraries

Peptide coding gene fragments from target genes were composed of the DNA coding sequence for all 40 mer amino acids from the genes/mutants listed in TABLE 1 below:

TABLE 1

| Genes | # of Mutants |
|---|---|
| Controls | |
| HPRT1 | NA |
| GFP | NA |
| Apoptosis | |
| CASP8 | 3 |
| Cell Cycle | |
| CDKN2A | 3 |
| RB1 | 1 |
| Chromatin Histone Modifiers | |
| EP300 | 8 |
| KMT2C | 2 |
| SETD2 | 5 |
| Genome Integrity | |
| CHEK2 | 6 |
| TP53 | 195 |
| MAPK Signaling | |
| BRAF | 16 |
| HRAS | 11 |
| KRAS | 16 |
| MAP2K1 | 4 |
| NRAS | 9 |
| RRAS2 | 2 |
| Metabolism | |
| IDH1 | 2 |
| IDH2 | 5 |
| NOTCH Signaling | |
| NOTCH1 | 3 |
| Other Signaling | |
| CDH1 | 4 |
| GNA11 | 4 |
| GNAQ | 4 |
| KEAP1 | 9 |
| PTPN11 | 15 |
| RAC1 | 8 |
| RHOA | 16 |
| PI3K Signaling | |
| AKT1 | 3 |
| PIK3CA | 12 |
| PIK3R1 | 11 |
| PPP2R1A | 13 |
| Protein Homeostasis | |
| FBXW7 | 13 |
| VHL | 24 |
| SPOP | 2 |
| RNA Abundance | |
| DDX3X | 3 |
| DICER1 | 9 |
| ZFP36L2 | 1 |
| RTK Signaling | |
| EGFR | 19 |
| ERBB2 | 3 |
| ERBB3 | 3 |
| ERBB4 | 3 |
| FGFR2 | 3 |
| FGFR3 | 2 |
| FLT3 | 2 |
| KIT | 9 |
| MET | 4 |
| RASA1 | 1 |
| Splicing | |
| SF3B1 | 16 |
| TGFB Signaling | |
| SMAD2 | 2 |
| SMAD4 | 31 |
| TGFBR2 | 3 |
| TOR Signaling | |
| MTOR | 9 |
| RHEB | 1 |
| Transcription Factor | |
| NFE2L2 | 9 |
| RUNX1 | 2 |
| Wnt/B-Catenin Signaling | |
| CTNNB1 | 15 |

For fitness screens the 5′ and 3′ ends of each gene fragment were modified to contain a start and stop codon, as well as ~20 bp of DNA homologous to the expression plasmid for downstream Gibson cloning.

### Cancer Driver Gene Fragment Cloning

Peptide coding gene fragment libraries were synthesized as pooled single stranded oligonucleotides by Custom Array. These oligonucleotides were then PCR amplified to generate double stranded gene fragments compatible with Gibson cloning. 50 ml PCR reactions were set up with 25 ng of pooled oligonucleotide template and 2.5 ml of primers PEP_1 and PEP_2 (10 mM). The thermal cycler was programmed to run at 95 C for 3 minutes, followed by 12 cycles of 98 C for 20 seconds, 65C for 15 seconds, and 72C for 45 seconds. This was followed by a final 5-minute extension at 72 C. PCR products were then purified.

### Fitness Screening in Mammalian Cell Lines

Hs578T cells and MDA-MB-231 cells were cultured in DMEM media supplemented with 10% FBS. Cells were transduced with the peptide coding gene fragment library at an MOI <0.3 to ensure each cell received a single construct. Viral transduction was performed in media containing 8 mg/ml polybrene to improve transduction efficiency. For each cell line, screening was conducted with two biological replicates. 24 hours after transduction the cell culture media was changed back to DMEM without polybrene supplementation.

48 hours after transduction, the cell culture media was changed to DMEM containing puromycin to select for transduced cells. 2 mg/ml puromycin was used to select the Hs578T cells, and 3.5 mg/ml puromycin was used to select the MDA-MB-231 cells. In the pilot screens, more than 6,000,000 cells (from each cell line) were transduced to ensure greater than 1000-fold coverage of the library. The cells were cultured for 14 days after transduction, and genomic DNA was isolated at days 3 and 14. For the larger screens, the number of cells transduced was scaled up accordingly.

### HTS Library Preparation and Sequencing

Peptide coding gene fragments for each time point and replicate were then amplified from the genomic DNA. The fragments serve as their own barcodes for downstream abundance calculations. Illumina compatible libraries were prepared using 2.5 ml of primers PEP_3 and PEP_4 (10 mM) per 50 ml reaction. For each sample (i.e. time point and replicate) from the pilot library, 10 separate 50 ml PCR reactions with 4 mg of gDNA each (40 mg total) were performed to ensure adequate library coverage. Thermal cycling parameters were identical to those used to amplify the gene fragment oligos, with the exception that the gDNA required 26 cycles to amplify. Sequencing was performed and samples were indexed. Greater than 500-fold sequencing depth was used to ensure accurate abundance quantitation. For the larger libraries, the number of PCR reactions was scaled to process 300 mg of total gDNA per timepoint and replicate. The larger libraries were then sequenced with 100-bp paired end reads.

### Example 2—Curation of Peptide Hits from Initial Screen

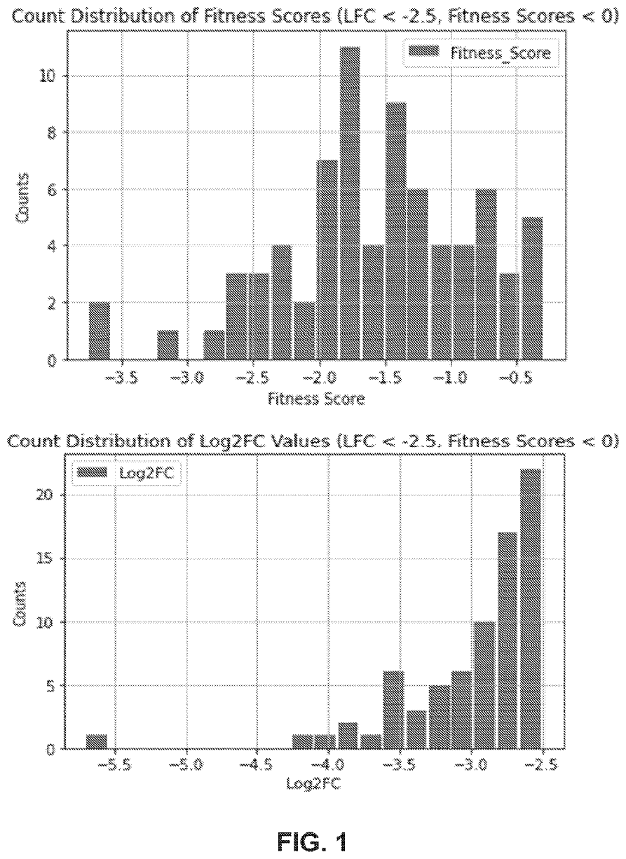

Hits selected in the initial screen of Example 1 were curated to identify hit alpha helical peptides for further engineering to improve binding affinity and enable more effective drug-like function. To this end, the data from Example 1 was filtered to only have peptide fragments that had a Log 2FC of less than 2.5 and a computed fitness score of less than 0, which selects for peptides with a negative impact on cell fitness. The peptides were then mapped to the Uniprot database for their gene names, annotated regions for containing alpha helices and regions that had crystal structures available. The focus was primarily on extracting alpha helices as it was needed for engineering staples and crystal structure availability as it was needed for the downstream algorithms for optimizing binding affinity. Additionally, the Interactome INSIDER database was used to extract experimentally curated and computationally predicted interacting residues/regions of proteins. The peptide fragments filtered earlier with a negative impact on cell fitness based on our screen was then filtered to ensure crystal structure availability, include alpha helical region, and span the predicted interacting residue regions. The count distribution of the peptides that passed these filters are shown in FIG. 1.

Based on the count distribution above, the fitness score cutoff was adjusted to less than 0.5 and a final list of 66 peptide fragments for 18 genes were compiled. With the goal of picking a best binder for each gene for further binding affinity optimization, the RCSB database was then manually searched for each peptide fragment to filter fragments that had co-crystal structures with different binding partners. Those that did not have crystal structures with binding partners and/or had too many unmodelled residues were dropped. For each peptide fragment that spanned the same region and shared the same structure file for its binding partners, the most negative Log 2FC fragment was chosen. The final list of peptides consisted of 11 peptide fragments for 11 genes summarized in TABLE 2. The alpha coverage is the number of residues that are part of an alpha helix contained in the 40-mer peptide and the PDB IDs are the ID of the structure files that contains both the target protein and a binding partner.

TABLE 2
Summary table of final list of manually filtered alpha helical hit peptide fragments.

| | | Uniprot | Fitness_ | P_ | | | | Alpha | PDB |
|---|---|---|---|---|---|---|---|---|---|
| Genc | AA | ID | Score | Value | Peptide | Log2FC | FDR | Coverage | IDs |
| RB1 | 521 | P06400 | −2.79834 | 0.000613 | RB1_521 | −2.8241 | 1.80E−06 | 29 | 1N4M |
| CHEK2 | 389 | O96017 | −1.777 | 0.000613 | CHEK2_389 | −2.7097 | 3.66E−19 | 24 | 3I6U |
| MDM2 | 85 | Q00987 | −2.05646 | 0.000613 | MDM2_85 | −3.5492 | 6.41E−14 | 11 | 1T4E, |
| | | | | | | | | | 2MPS, |
| | | | | | | | | | 4HFZ |
| RAF1 | 78 | P04049 | −0.72041 | 0.010181 | RAF1_78 | −3.5166 | 1.55E−12 | 14 | 1C1Y, |
| | | | | | | | | | 4G0N |
| RRAS2 | 99 | P62070 | −1.69274 | 0.000613 | RRAS2_99 | −3.8967 | 4.66E−09 | 16 | 2ERY |
| RAC1 | 62 | P63000 | −1.38853 | 0.000613 | RAC1_62 | −4.0504 | 5.95E−08 | 21 | 2FJU, |
| | | | | | | | | | 2RMK, |
| | | | | | | | | | 4YON, |
| | | | | | | | | | 3B13, |
| | | | | | | | | | 3SU8 |
| PIK3CA | 943 | P42336 | −1.70565 | 0.000613 | PIK3CA_943 | −2.6908 | 4.70E−16 | 18 | 4OVU |
| PIK3R1 | 442 | P27986 | −2.61004 | 0.000613 | PIK3R1_442 | −3.8354 | 7.34E−23 | 40 | 4OVU |
| VHL | 162 | P40337 | −0.77206 | 0.004068 | VHL_162 | −2.5355 | 0.000168 | 29 | 4WQO |
| DDX3X | 243 | O00571 | −1.4947 | 0.000613 | DDX3X_243 | −2.7302 | 3.74E−08 | 18 | 5E7I |
| CTNNB1 | 303 | P35222 | −0.57131 | 0.029214 | CTNNB1_303 | −2.5452 | 5.28E−12 | 30 | 1TH1, |
| | | | | | | | | | 1JDH, |
| | | | | | | | | | 3TX7 |

### Example 3—Mutagenesis to Improve Binding Affinity

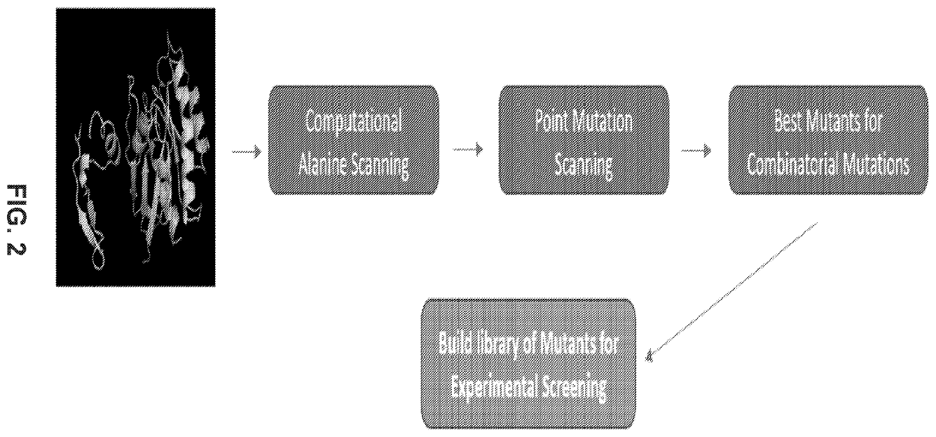

With the list of alpha helical hit peptides extracted from the fitness screen data in Example 2, a workflow was developed to improve the binding affinity of these peptides computationally (FIG. 2).

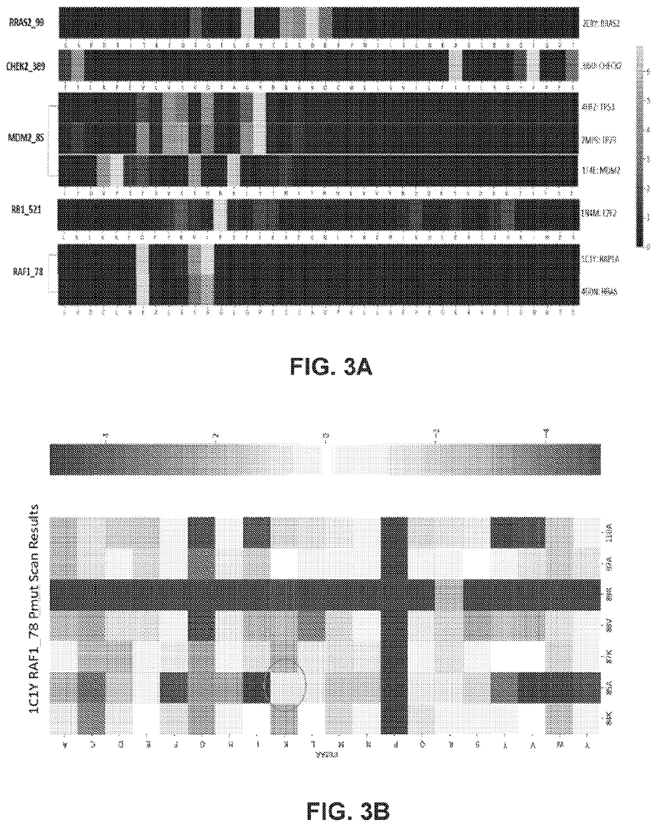

The workflow consists of starting with the structure file (PDB) of an identified alpha helical 40-mer peptide binding to an interacting protein. The computational alanine scanning algorithm is then used to identify residues in the binding interface by calculating the binding energy of the structure after mutating each residue at the interface to alanine. The residue mutated that destabilizes the binding is predicted to be a residue that is important for binding. With the list of residues important for binding, in silico site saturation mutagenesis is conducted and binding affinity differences is evaluated with the point mutation scanning algorithm (pmut scan) to identify mutants with the highest binding affinity. This was done with the built in pmut scan method in the Rosetta software. An example of using this workflow with an identified alpha helical 40-mer peptide derived from RAF1 (RAF1_78) is shown in FIG. 3A and FIG. 3B.

FIG. 3A depicts a heatmap of computational alanine scanning results of identified alpha helical 40-mer peptides (5 out of 11) binding to interacting proteins. The Protein Data Bank Identifier (PDB ID) followed by the interacting protein is labeled on the right side. The amino acid sequence of the 40-mer peptide is labeled on the bottom and the corresponding computational alanine binding energy with the interacting protein, delta-delta-G (ddG), is plotted. The higher the value is, the more likely that particular residue is important for binding. FIG. 3B is a heatmap of the point mutation scanning result of RAF1_78 binding to RAP1A (PDB ID: 1C1Y). The residues for conducting site saturation mutagenesis are identified via the results from computational scanning and is labelled on the bottom. The mutated amino acids are labelled to the left and the ddG of the final mutated structure is plotted. The more negative the value is, the higher the binding affinity is between the 40-mer peptide-interacting protein complex. The circled residue is A85K and has been identified as increasing binding affinity between RAF1 and HRAS.

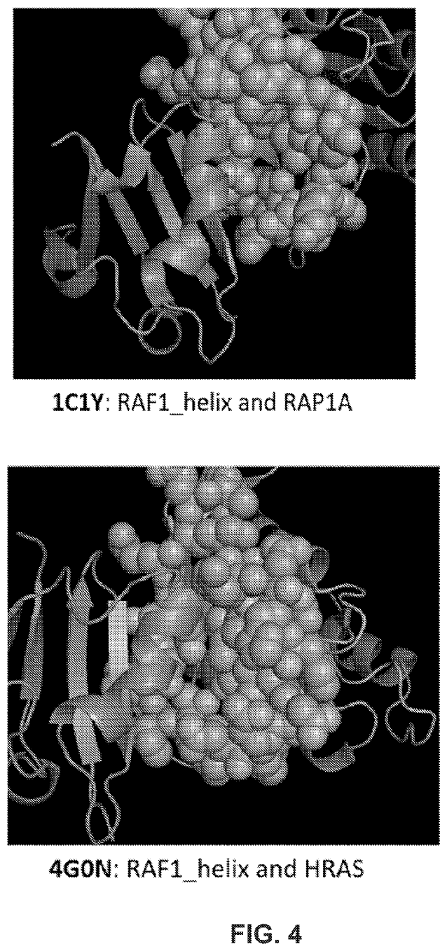

FIG. 4 depicts the structure of the interaction between RAF1 and an interacting protein (RAP1A or HRAS).

### Example 4—RAC1 and RAF1 Helix Engineering and Stapling

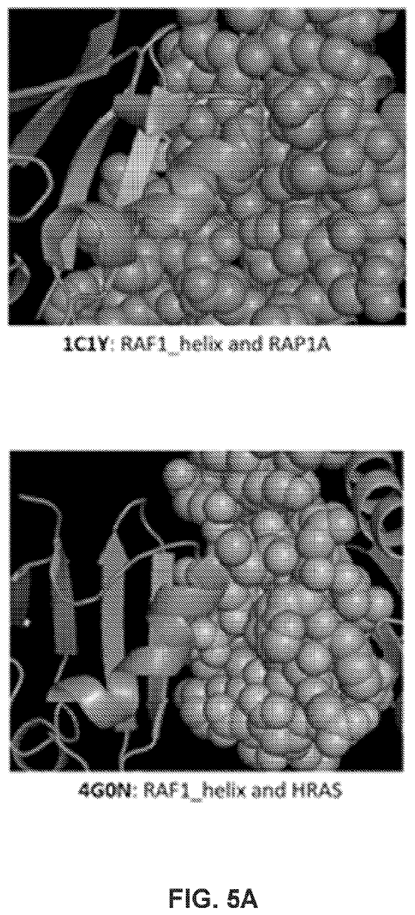

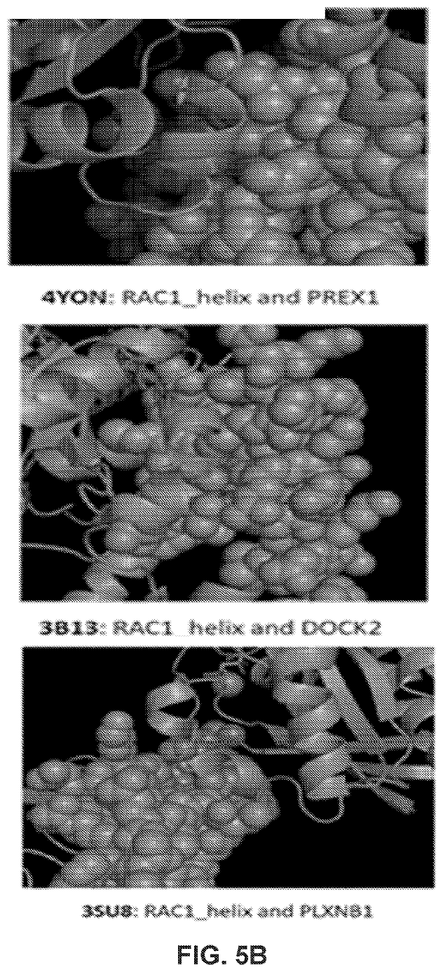

RAC1 and RAF1 were identified from the workflow of Example 1 and Example 2 regarding identifying alpha-helical 40-mer peptides from the peptide screens and the workflow of Example 3 was performed on select identified alpha-helical 40-mer peptides. During the step of manually verifying peptides and their co-crystal structures, RAC1-61 and RAF1-78 were two alpha-helical 40-mer peptides that had a region of alpha helix that fit the binding interface with their interacting proteins really well. Based on their co-crystal structures as shown in FIG. 5A-5B, the 40-mer peptide was shortened to only span the alpha helical region. Additionally, staples were designed to enable cell-penetrating capabilities of these peptides to improve their drug-like function. Shorter staples (i, i+3) and longer (i, i+7) staples were both evaluated. The longer staple (i, i+7) was chosen for the subsequent examples.

The final sequence details of the RAC1 and RAF1 helical peptides for stapling are indicated in TABLE 3 below:

TABLE 3
Representative Engineered Therapeutic Peptides

| Name | Sequence | Stapled Sequence |
|---|---|---|
| RAFIhelix (i, i + 7 staple) | SLHDCLMKALKVR (SEQ | SL-R8-DCLMKA-S5-KVR |
| | ID NO: 1) | (SEQ ID NO: 4) |
| RAFIhelix A85K mutant | SLHDCLMKKLKVR (SEQ | SL-R8-DCLMKK-S5-KVR |
| (i, i + 7 staple) | ID NO: 2) | (SEQ ID NO: 5) |
| RACIhelix (i, i + 7 staple) | EDYDRLRPLSYPQT (SEQ | EDY-R8-RLRPLS-S5-PQT |
| | ID NO: 3) | (SEQ ID NO: 6) |

R-octenylalanine (R8) and S-pentenylalanine (S5) are non-naturally occurring amino acid residues that allow the formation of the hydrocarbon staple. The image below discloses SEQ ID NOS 8-10, respectively, in order of appearance.

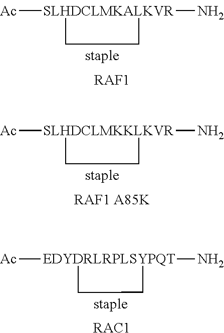

### Example 5—In Vitro Experiments

### Activity

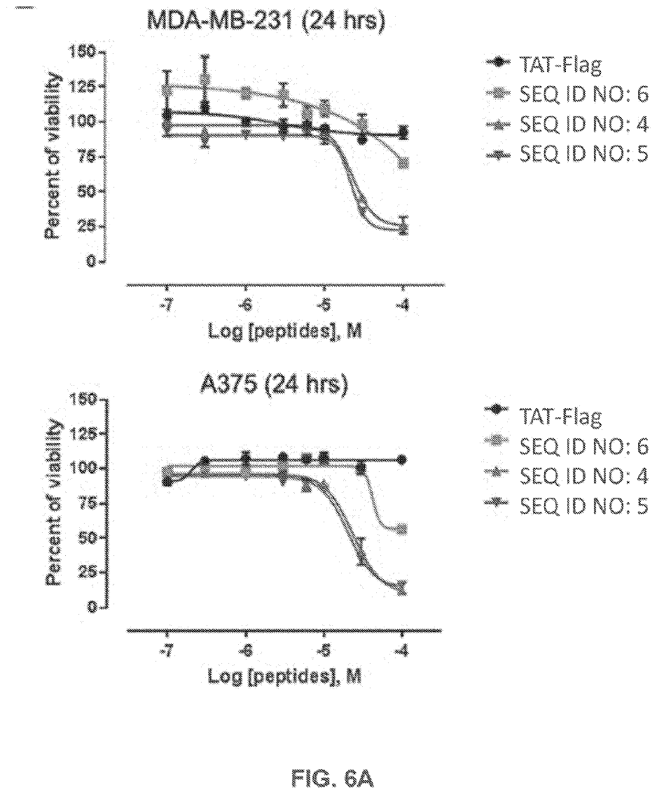

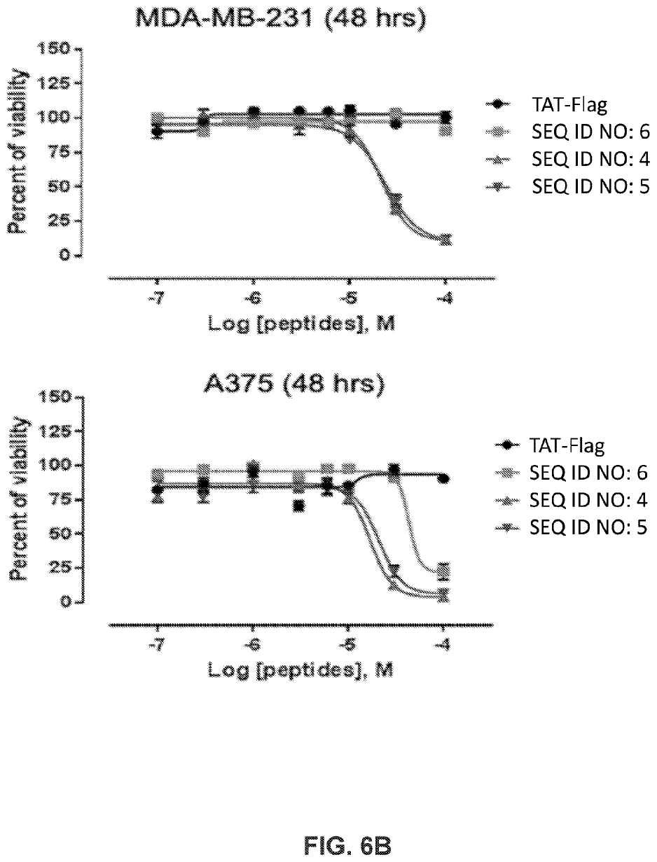

MDA-MB-231 and A375 cell lines were treated with various doses of either TAT-Flag peptide (control), or peptide of SEQ ID NO: 4, SEQ ID NO: 5, or SEQ ID NO: 6 (0.1 μM, 0.3 μM, 1 μM, 3 μM, 10 μM, 30 μM, 100 μM). FIG. 6A and FIG. 6B depicts dose response curves for each peptide. Cell viability was measured at 24-hours (FIG. 6A) and 48-hours (FIG. 6B) post treatment using the colorimetric assay Cell Counting Kit-8 (CCK8). TAT-Flag peptide treatment did not affect cell viability at 24 and 48 hours, while peptide of SEQ ID NO: 4 or SEQ ID NO: 5 affected cell viability of both cell lines, and peptide of SEQ ID NO: 6 only affected cell viability of A375 cells. At 48 hours, peptide of SEQ ID NO: 6 displayed an IC50=41.8 μM with a maximum effect of 77.8% cell death in A375 cells, whereas MDA-MB-231 cell survival is not significantly altered by peptide of SEQ ID NO: 6. A375 and MDA-MB-231 cells responded to both peptide of SEQ ID NO: 4 and peptide of SEQ ID NO: 5 in a similar fashion as shown by the overlapping dose response curves. Therefore, there was no differential bioactivity on cell death between WT and A85K RAF1 stapled peptides. Between MDA-MB-231 and A375 cell lines a slight difference of sensitivity towards both peptide of SEQ ID NO: 4 and peptide of SEQ ID NO: 5 was observed. Indeed, peptide of SEQ ID NO: 4 and peptide of SEQ ID NO: 5 had respectively an IC₅₀=21.2-23.2 μM with a maximum effect of 89.6% on cell death in MDA-MB-231 cells and IC₅₀=16.9-20.8 μM with a maximum effect of 96.2% on cell death in A375 cells.

### Impact of Serum Starvation

A375 cells were starved by replacing the normal media (DMEM/10% FBS) by a media without FBS (DMEM/0% FBS) for 24 hours. Then, cells were treated with peptide of SEQ ID NO: 4 at 10 μM for 48 hours and cell viability was quantified using colorimetric assay CCK8. Under normal condition (10% FBS), treatment with SEQ ID NO: 4 peptide decreased cell viability by 16.6% compared to the vehicle condition. When A375 were deprived of FBS, treatment with SEQ ID NO: 4 peptide decreased cell viability by 49.6% compared to the vehicle under starved condition. Thus, when A375 cells were starved, sensitivity to SEQ ID NO: 4 peptide was increased by ~3 fold.

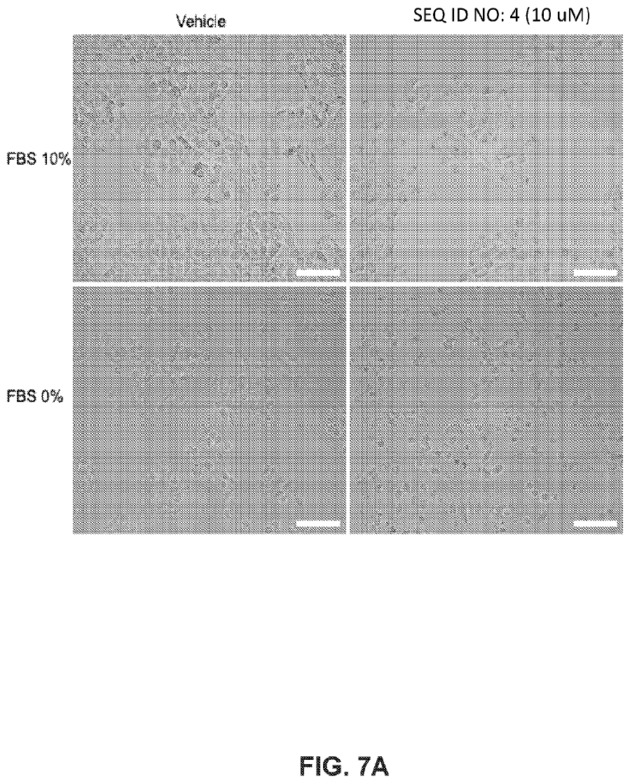

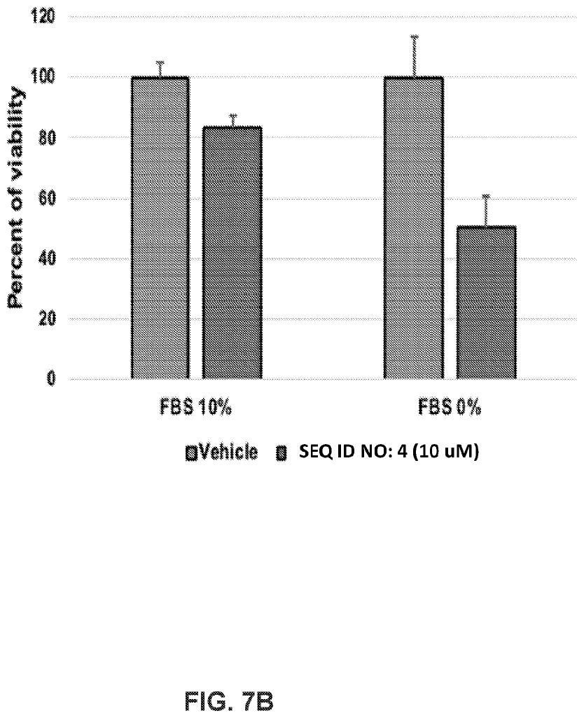

FIGS. 7A and 7B show starvation potentiates the anti-proliferative effect of SEQ ID NO: 4 peptide. In FIG. 7A, A375 cells were treated for 48-hours with SEQ ID NO: 4 peptide at 10 μM or vehicle (PBS) with either 10% FBS or 0% FBS (starvation condition). Bright field images were taken with 20× magnification objective. Scale bar=100 μm. FIG. 7B shows quantification of cell viability measured by CCK8.

### Example 6—Tumor Experiments

This example shows tumor growth arrest in vivo after administration of SEQ ID NO: 4 peptide in three different sets of tumor efficacy experiments.

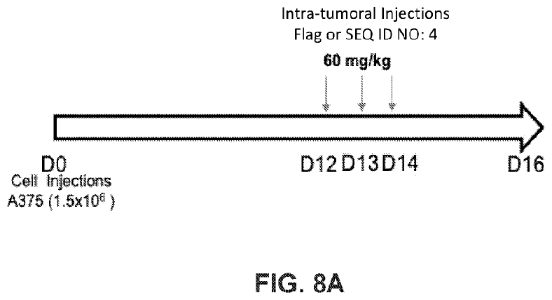

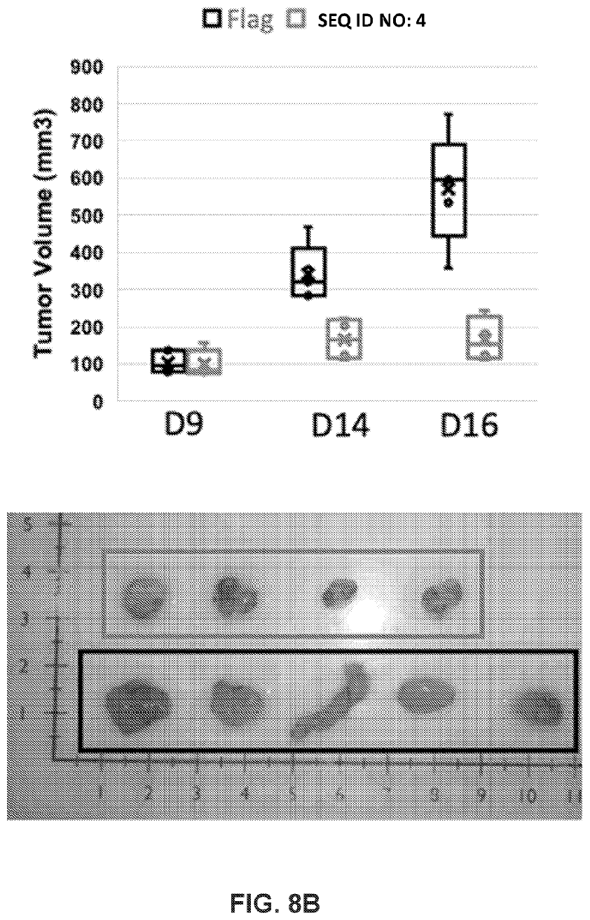

FIG. 8A shows a timeline for a first set of tumor efficacy experiments. 1.5 million A375 cells in 50% Matrigel were injected subcutaneously in the back of NOD-scid gamma mice. At Day 9 post-injection, tumor volumes reach an average of 102±31 mm³ and then mice were split into two groups with similar tumor size distribution. At Day 12 post-injections, tumors were injected with either SEQ ID NO: 4 peptide, or Flag peptides (Flag) resuspended in PBS at 60 mg/kg. Each mouse was treated with 1 intra-tumoral injection every day for 3 days. FIG. 8B depicts the change in tumor volume observed with SEQ ID NO: 4 peptide or a Flag (Flag) control. At Day 14, a significant difference of the tumor volume between Flag (342±76 mm³) and SEQ ID NO: 4 peptide (165±55 mm³) treated mice was observed. At Day 16, tumors of the Flag group kept growing (572±149 mm³) whereas tumor volume of the SEQ ID NO: 4 peptide treated group remained steady compare the previous time point at Day 14. Thus, treatment with SEQ ID NO: 4 peptide led to tumor growth arrest in vivo.

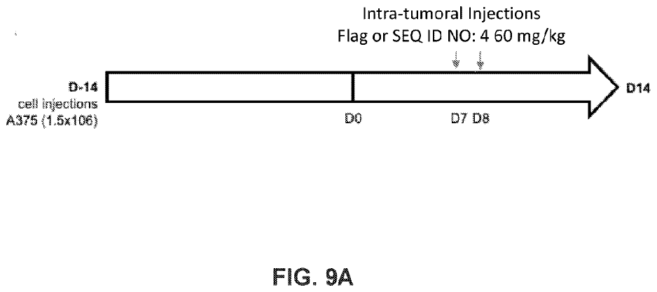

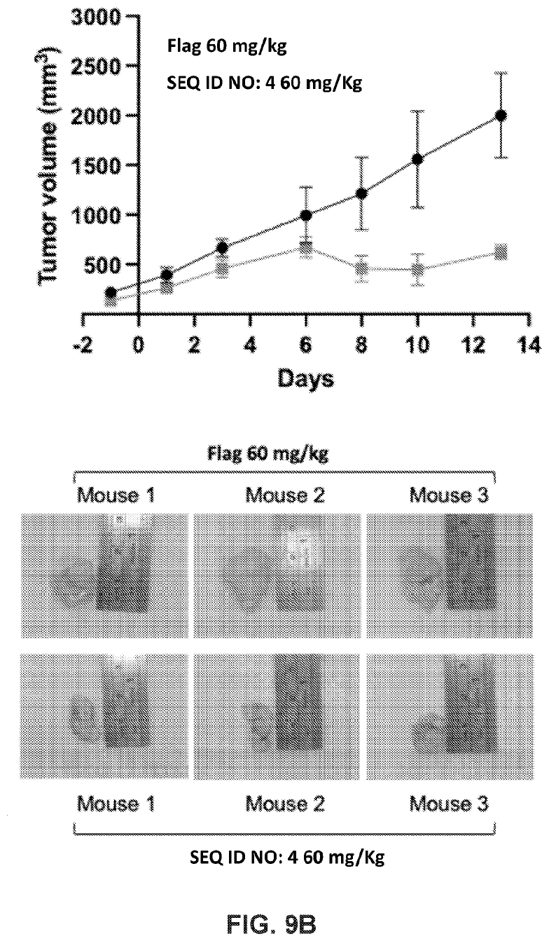

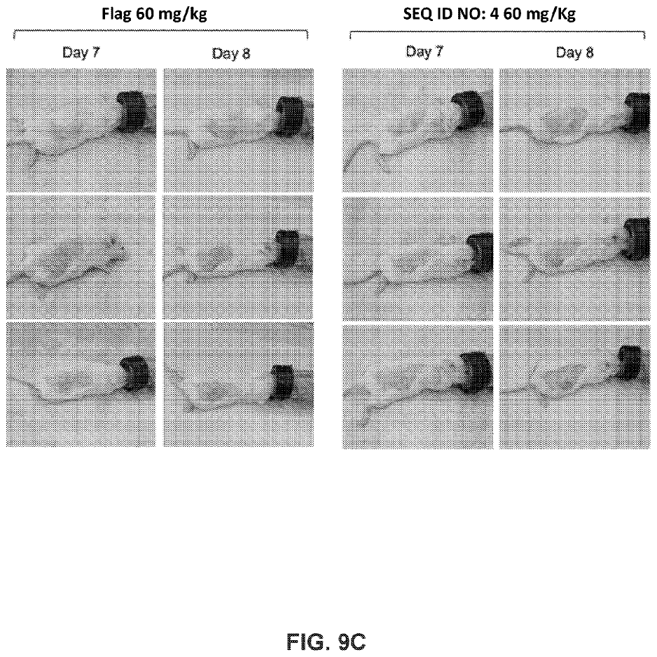

FIG. 9A shows a timeline for a second set of tumor efficacy experiments. 1.5 million A375 cells in 50% Matrigel were injected subcutaneously in the back of NOD-scid gamma mice. 13 days later (D-1), tumor volumes reached an average of 149±27 mm³ and mice were divided into two cohorts, Flag 60 mg/kg and SEQ ID NO: 4 peptide 60 mg/kg. Tumor volumes were measured every two days. At day 6, tumors volume in both groups were greater than 500 mm³. At day 7 and 8, tumors were treated by intratumoral injections with either Flag or SEQ ID NO: 4 peptides at 60 mg/kg. Each mouse received two injections in total. Strikingly, the day after the first injection (day 8), tumors appeared to flatten down in SEQ ID NO: 4 peptide treated group but not in the Flag treated group (FIG. 9C). This observation was confirmed by tumor volume measurement on day 8. In the Flag treated group, tumors kept growing after the first injection reaching 2001 mm³ on day 13, while tumors in SEQ ID NO: 4 peptide treated group appeared to stop growing (FIG. 9B).

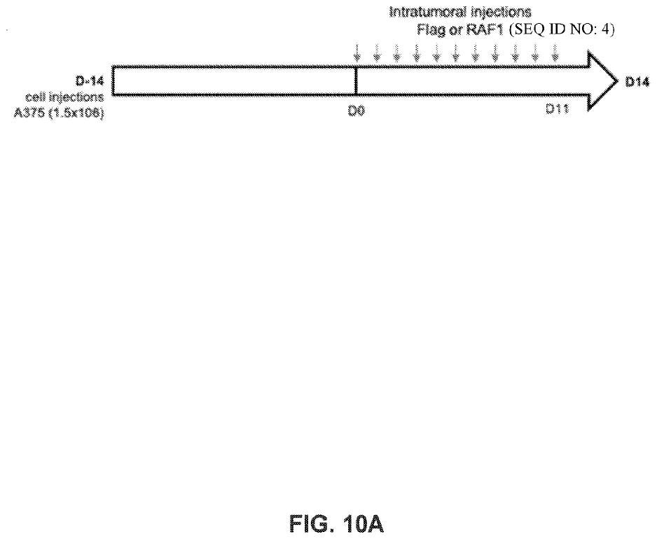

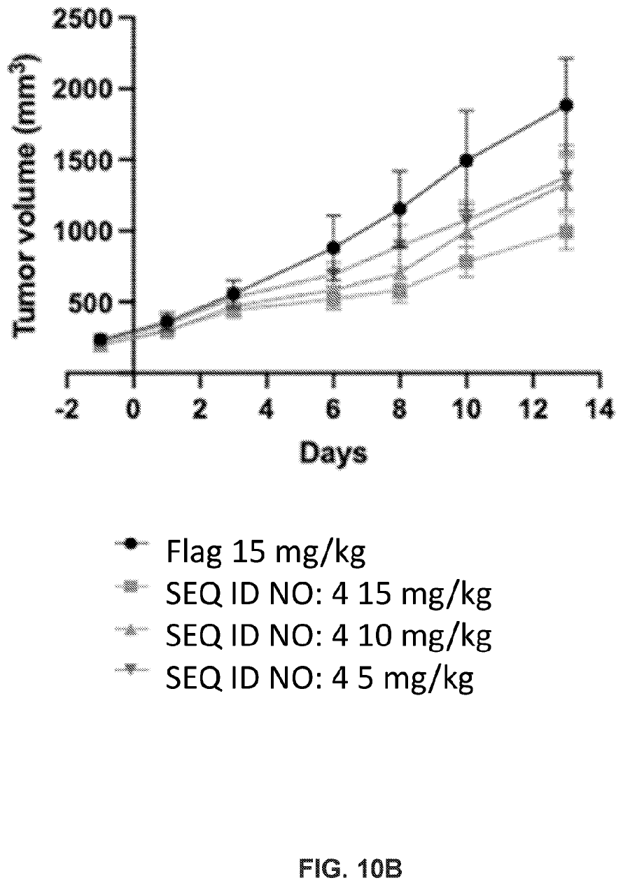

FIG. 10A shows a timeline for a third set of tumor efficacy experiments. 1.5 million A375 cells in 50% Matrigel were injected subcutaneously in the back of NOD-scid gamma mice. 13 days later (D-1), tumor volumes reached an average of about 200 mm³ and mice were divided into four cohorts: Flag 15 mg/kg, and SEQ ID NO: 4 peptide at 15 mg/kg, 10 10 mg/kg, or 5 mg/kg. Mice were treated with the control (Flag) or SEQ ID NO: 4 peptides at the indicated dosage every day for 11 days. Tumor volumes were measured about every two days. At day 6, tumors volume in all groups were greater than 500 mm³. At day 13 the mice receiving the SEQ ID NO: 4 peptide at the indicated dosage had reduced tumor volumes as compared to the control group (Flag peptide), as shown in FIG. 10B.

The tumors in the first set of tumor efficacy experiments and the second set of tumor efficacy experiments were treated with dose of 60 mg/kg but at different stage of tumor growth. The tumors in the first set of tumor efficacy experiments were treated once reaching a volume greater than 100 mm³ versus tumors in the second set of tumor efficacy experiments were treated tumors were greater than 500 mm³. In both sets of experiments, SEQ ID NO: 4 peptide stopped the tumor growth, and the effect on the tumor shape could be observed one day after the injection. Similarly, in the third experiment mice treated daily with SEQ ID NO: 4 peptide had reduced tumor volume in all tested conditions (5-15 mg/kg) compared to the control. In conclusion, SEQ ID NO: 4 peptide had in vivo bioactivity and reduced tumor growth of human melanoma cells.

### Example 7—Tumor Experiments

This example describes assessment of tumor growth in vivo after administration of SEQ ID NO: 1 peptide, SEQ ID NO: 2 peptide, SEQ ID NO: 3 peptide, SEQ ID NO: 4 peptide, SEQ ID NO: 5 peptide, or SEQ ID NO: 6 peptide in different KRAS mutant tumor models.

A pancreatic tumor cell line having a G12V KRAS mutation or G12C KRAS mutation in 50% Matrigel are injected subcutaneously in the back of NOD-scid gamma mice. Tumor volumes are allowed to grow and then mice are split into two groups with similar tumor size distribution. Tumors are injected either intra-tumoral or intraperitoneally with SEQ ID NO: 1 peptide, SEQ ID NO: 2 peptide, SEQ ID NO: 3 peptide, SEQ ID NO: 4 peptide, SEQ ID NO: 5 peptide, SEQ ID NO: 6 peptide or a control peptide resuspended in PBS at 5 mg/kg, 15 mg/kg, 60 mg/kg, or 240 mg/kg. Each mouse is treated with 1 intra-tumoral injection every 3 days. Tumor growth is measured.

A lung tumor cell line having a G12V KRAS mutation or G12C KRAS mutation in 50% Matrigel are injected subcutaneously in the back of NOD-scid gamma mice. Tumor volumes are allowed to grow and then mice are split into two groups with similar tumor size distribution. Tumors are injected either intra-tumoral or intraperitoneally with SEQ ID NO: 1 peptide, SEQ ID NO: 2 peptide, SEQ ID NO: 3 peptide, SEQ ID NO: 4 peptide, SEQ ID NO: 5 peptide, SEQ ID NO: 6 peptide or a control peptide resuspended in PBS at 5 mg/kg, 15 mg/kg, 60 mg/kg, or 240 mg/kg. Each mouse is treated with 1 intra-tumoral injection every 3 days. Tumor growth is measured.

A colorectal tumor cell line having a G12V KRAS mutation or G12C KRAS mutation in 50% Matrigel are injected subcutaneously in the back of NOD-scid gamma mice. Tumor volumes are allowed to grow and then mice are split into two groups with similar tumor size distribution. Tumors are injected either intra-tumoral or intraperitoneally with SEQ ID NO: 1 peptide, SEQ ID NO: 2 peptide, SEQ ID NO: 3 peptide, SEQ ID NO: 4 peptide, SEQ ID NO: 5 peptide, SEQ ID NO: 6 peptide or a control peptide resuspended in PBS at 5 mg/kg, 15 mg/kg, 60 mg/kg, or 240 mg/kg. Each mouse is treated with 1 intra-tumoral or intraperitoneal injection every 3 days. Tumor growth is measured.

### Example 8—In Vitro Selection of Tumor Cell Lines

This example shows in vitro testing of SEQ ID NO: 4 peptide potency against KRAS-mutant cell lines and selection of cell lines for in vivo testing.

Prior to in vitro testing, the SEQ ID NO: 4 peptide was determined to have solubility in PBS of up to at least 100 mg/mL. The peptide was prepared by a contract manufacturer and lyophilized prior to being reconstituted. The acetate salt of the SEQ ID NO: 4 peptide and the control peptide was used in this example and the examples described below. SEQ ID NO: 4 was tested in a 10-point dose curve at a 1:2 dilution with a top concentration of 100 μM and a 72 hour endpoint against different KRAS-mutant cell lines. A cell titer-glo assay was used to determine the potency of the peptide against different cell lines. The summary of the potency of SEQ ID NO: 4 against different KRAS-mutant cell lines is shown on TABLE 4. TABLE 4 shows the mutant cell line tested, the tumor type of the mutant cell line, the KRAS mutation status, the half maximal effective concentration (EC50) of the TAT-Flag control (GRKKRRQRRRPQDYKDDDDK SEQ ID NO: 7), the EC50 of the SEQ ID NO: 4 peptide, and if the cell line was selected for an in vivo study (Example 9).

TABLE 4
In vitro potency against KRAS-mutant Cell Lines

| | | | | SEQ ID | Selected |
|---|---|---|---|---|---|
| | | | | NO: 4 | for in |
| | | KRAS | TAT-Flag | EC50 | vivo |
| Cell Line | Tumor type | status | EC50 | (μM) | study |
| SW480 | Colorectal | G12V | Not evaluable | 52.3 | Yes |
| SW620 | Colorectal | G12V | Not evaluable | Not | No |
| | | | | evaluable | |
| NCI-H727 | Lung | G12V | Not evaluable | 44.9 | No |
| NCI-H441 | Lung | G12V | Not evaluable | 9.18 | Yes |
| NCI-H2122 | Lung | G12C | Not evaluable | 45.3 | No |
| NCI-H358 | Lung | G12C | Not evaluable | 16.7 | Yes |
| A375 | Melanoma | WT | Not evaluable | 14.6 | No |
| MDA-MB- | Breast | G12D | Not evaluable | 161 | No |
| 231 | | | | | |

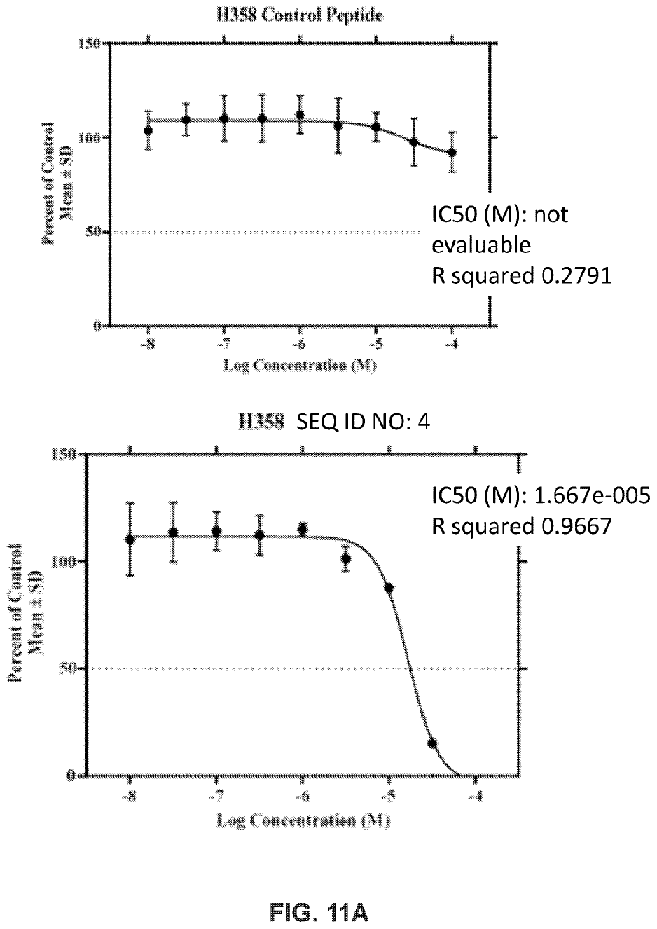

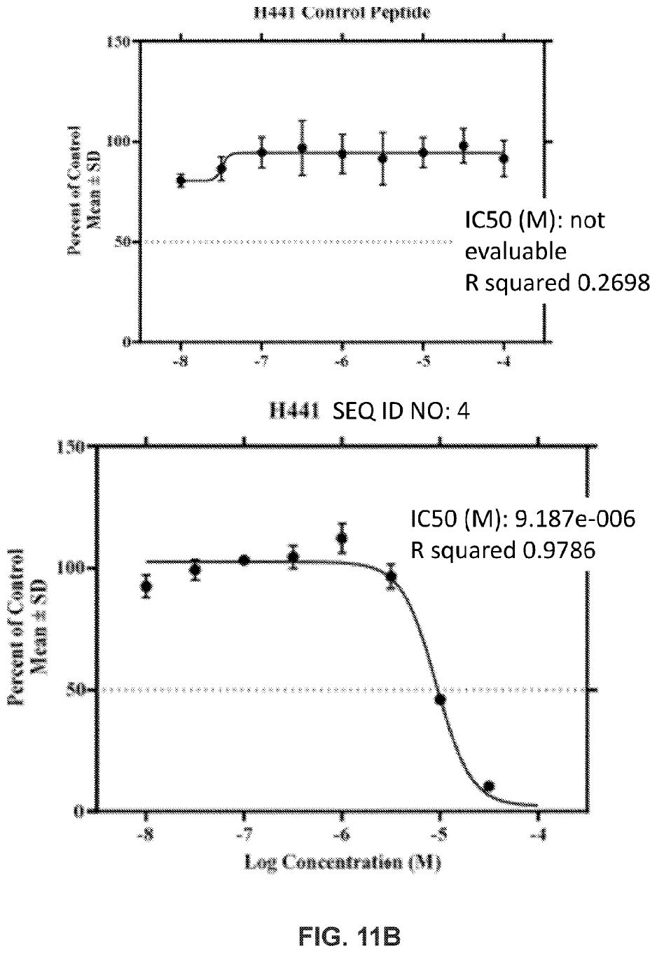

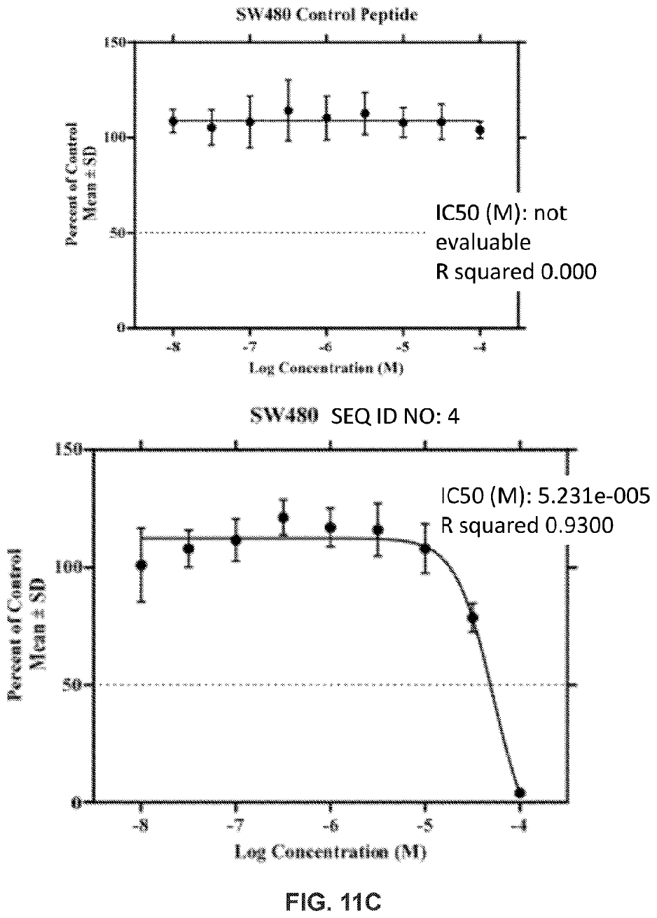

FIGS. 11A-11C show IC50 curves for SEQ ID NO: 4 peptide and a control peptide (TAT-Flag peptide, SEQ ID NO: 7) against different KRAS-mutant cell lines for a 72-hour endpoint and using a Cell Titer Glo assay. FIG. 11A shows an IC50 of 1.667e-005 M of the SEQ ID NO: 4 peptide against the NCI-H358 cell line; FIG. 11B shows EC50 of 9.187e-006 M of the SEQ ID NO: 4 peptide against the NCI-H441 cell line; and FIG. 11C shows EC50 of 5.231e-005 for the SEQ ID NO: 4 peptide against for the SW480 cell line. The NCI-H441 cell line (lung tumor), NCI-H358 cell line (lung tumor), and the SW480 cell line (colorectal tumor) were selected for further in vivo studies (See Example 9). Overall, SEQ ID NO: 4 peptide had EC50 values ranging from 9.18 μM to 161 μM against most of the KRAS mutant cell lines tested.

### Example 9—In Vivo Tumor Experiments

This example shows tumor growth arrest in vivo after administration of SEQ ID NO: 4 peptide in different mice tumor cell models.

The mice tumor models are summarized in TABLE 5, which shows the cell line used for the tumor model, the mouse strain used for the tumor model, the number of cells injected subcutaneously in the back of the mice, the volume of cells injected and the formulation used for injection, the tumor size at the randomization and start of treatment, and the max tumor volume observed. In all three tumor groups (NCI-H358, NCI-H441, and SW480) the mouse age at the start of the study was 8-12 weeks. The mouse strain for the NCI-H358 (H358) tumor model was Female CB.17 severe combined immunodeficient (SCID) mouse. The mouse strain for the NCI-H441 (H441) tumor model and the SW480 tumor model was the Female NCr mouse.

TABLE 5
In Vivo Tumor Model Details

| Cell line | | | Cell injection | | |
|---|---|---|---|---|---|
| for | | | volume | Tumor size at | Max |
| Tumor | Mouse | Cell | and | randomization/ | tumor |
| model | strain | number | formulation | treatment | volume |
| NCI- | Female | 1E+07 | 0.1 mL | 100-150 mm³ | >600 mm³ |
| H358 | CB.17 | | 50% Matrigel | | |
| | SCID | | subcutaneous | | |
| NCI- | Female | 5E+06 | 0.1 mL | 100-150 mm³ | >800 mm³ |
| H441 | NCr | | 50% Matrigel | | |
| | | | subcutaneous | | |
| SW480 | Female | 1E+07 | 0.1 mL | 100-150 mm³ | >1500 mm³ |
| | NCr | | 50% Matrigel | | |
| | | | subcutaneous | | |

In each tumor study (NCI-H358, NCI-H441, and SW480), the mice were separated into 4 groups with 8 mice per group. Group 1, was the vehicle alone; Group 2 was the TAT-Flag control peptide (SEQ ID NO: 7) at 4.5 mg per dose; Group 3 was low dose SEQ ID NO: 4 peptide at 1.5 mg per dose; and Group 4 was high dose SEQ ID NO: 4 peptide at 4.5 mg per dose. The peptides were administered intra-tumorally three times weekly and for four weeks (or until the tumor volume was too large). Measurements were recorded such as body weight, tumor volume, and tumor weight. A summary of the study design is shown in TABLE 6.

TABLE 6
In Vivo Study Design

| | | | Formu- | | | | |
|---|---|---|---|---|---|---|---|
| | | | lation | | Sche- | Dur- | Measure- |
| Group | N | Agent | dose | Route | dule | ation | ments |
| 1 | 8 | Vehicle | 50 uL | Intra- | Three | 4 | Body |
| | | | | tum- | times | weeks | weight, |
| | | | | oral | weekly | or | tumor |
| | | | | | | until | volume |
| | | | | | | tumor | (cali- |
| 2 | 8 | TAT- | 4.5 | | | vol- | pers), |
| | | Flag | mg | | | ume | tumor |
| | | (SEQ | | | | was | weight |
| | | ID | | | | too | at |
| | | NO: | | | | large | end- |
| | | 7) | | | | | point |
| 3 | 8 | SEQ | 1.5 | | | | |
| | | ID | mg | | | | |
| | | NO: | | | | | |
| | | 4 | | | | | |
| 4 | 8 | SEQ | 4.5 | | | | |
| | | ID | mg | | | | |
| | | NO: | | | | | |
| | | 4 | | | | | |

In the SW480 group 4 (SEQ ID NO: 4 peptide-high dose) dosing was stopped after Day 13 due to ulcerations and deaths in the group precluding statistical analysis. In the SW480 model, G1A6 (Vehicle animal 6), G3A4 (SEQ ID NO: 4 peptide-low dose animal 4), G3A8 (SEQ ID NO: 4 peptide-low dose animal 8), G4A1 (SEQ ID NO: 4 peptide-high dose animal 1), G4A3 (SEQ ID NO: 4 peptide-high dose animal 3), G4A5 (SEQ ID NO: 4 peptide-high dose animal 5), G4A6 (SEQ ID NO: 4 peptide-high dose animal 6), and G4A8 (SEQ ID NO: 4 peptide-high dose animal 8) died in the study. SW480 group animals were either found dead or reached a humane endpoint due to ulcerations. G4A5 and G4A6 were found dead and listed as not treatment-related as the ulcerations did not appear significant enough to induce death. G4A3 had a physical injury that prevented mobility so euthanasia was required. G4A8 and G4A1 were euthanized for extensive ulceration. In the H441 group, G4A4 (SEQ ID NO: 4 peptide-high dose animal 4) died in the study.

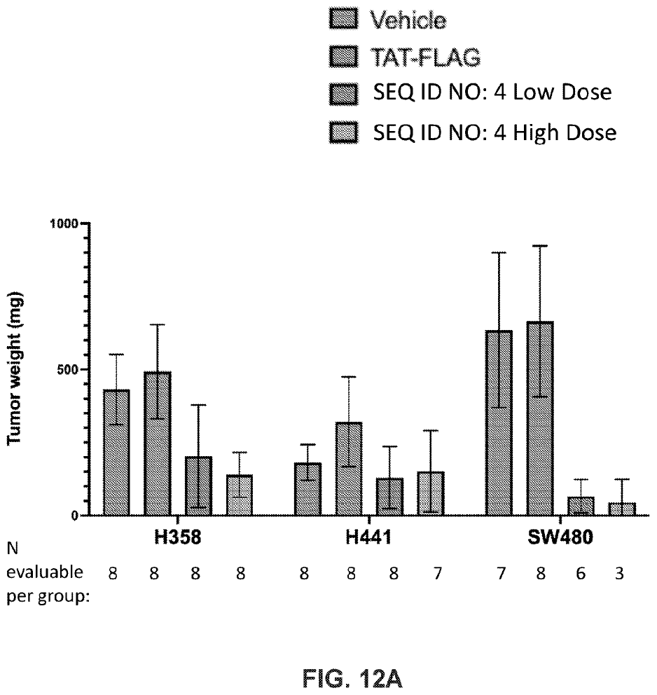

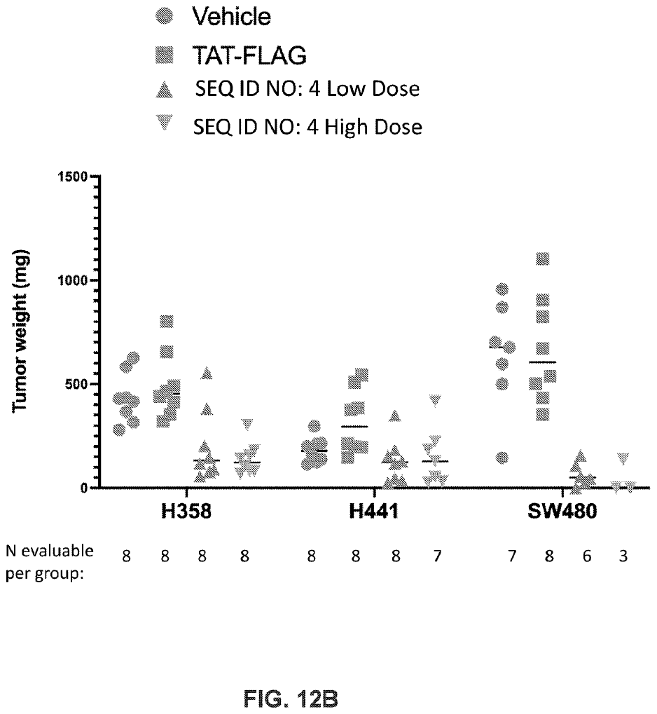

The tumor weight results after 4 weeks of treatment are shown in FIGS. 12A and 12B. The results show that treatment by injecting SEQ ID NO: 4 peptide reduced the tumor weight in the H358 and SW480 tumor mouse models as compared to the vehicle and control peptide. In the SW480 model, both the low dose (1.5 mg) and the high dose (4.5 mg) of SEQ ID NO: 4 peptide lowered the tumor weight of the animal. In the H358 model, the high dose (4.5 mg) of SEQ ID NO: 4 peptide lowered the tumor weight of the animal. These results show the peptide (SEQ ID NO: 4 peptide) is effective for treating lung and colorectal cancers.

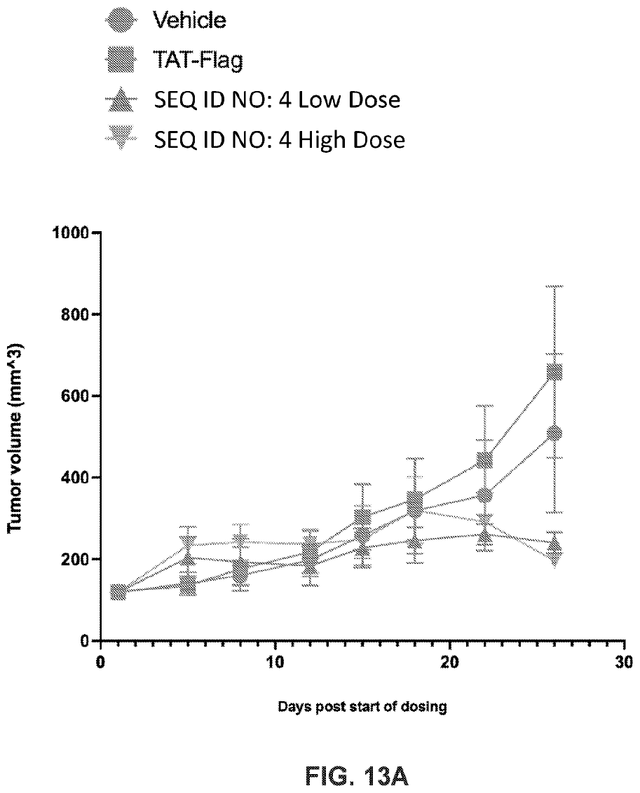

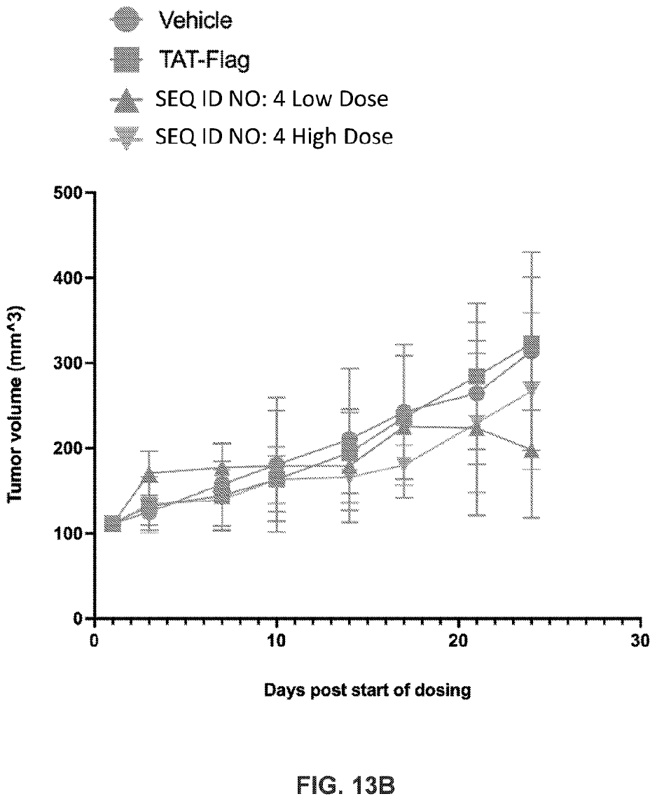

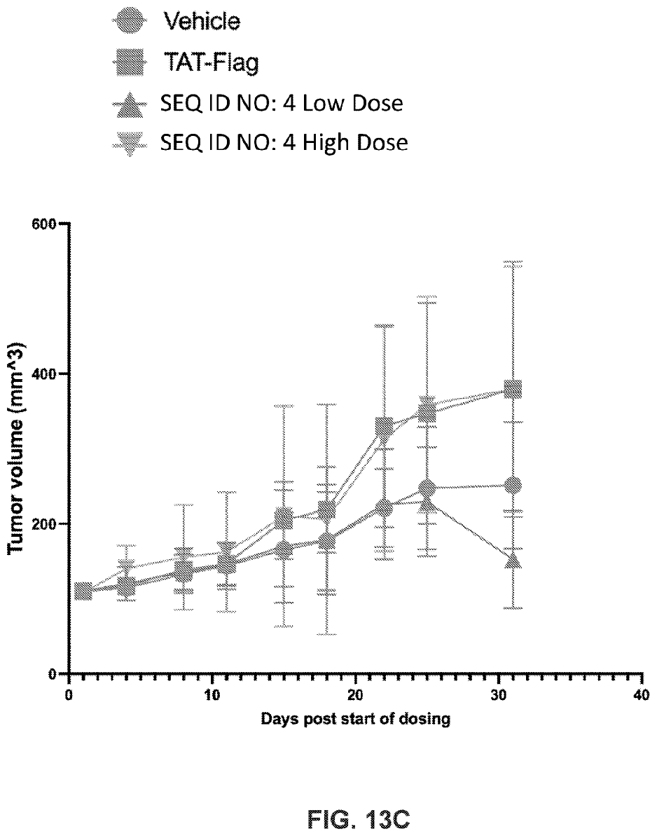

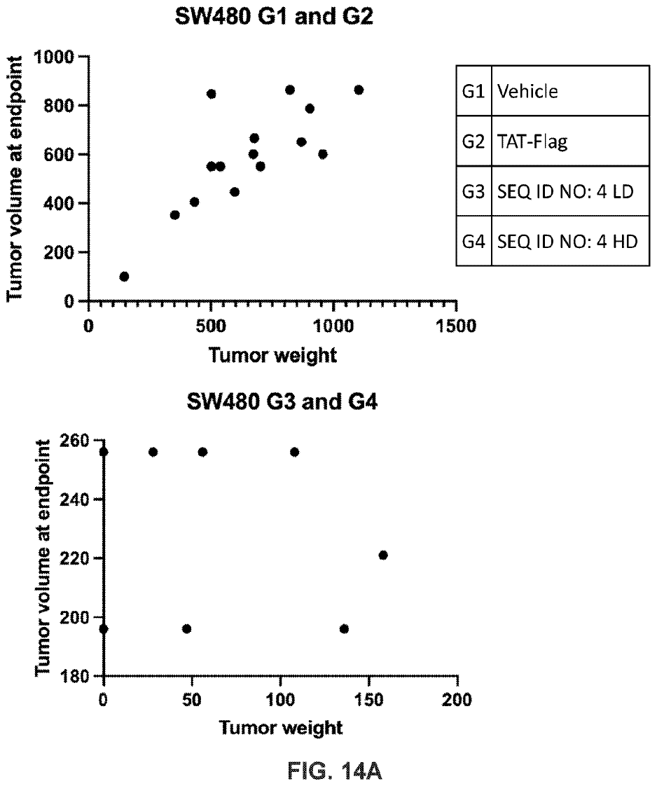

Mean tumor volumes for the groups were recorded throughout the study and are shown in FIGS. 13A-13C for the SW480 (FIG. 13A), NCI-H358 (FIG. 13B), and NCI-H441 (FIG. 13C) treatment groups. As shown in FIG. 13A, the tumor volume was decreased in the SEQ ID NO: 4 peptide low dose (1.5 mg) and high dose (4.5 mg) treatment groups in the SW480 tumor model as compared to the control treatment groups. This data shows the peptide (SEQ ID NO: 4 peptide) can reduce tumor volume.

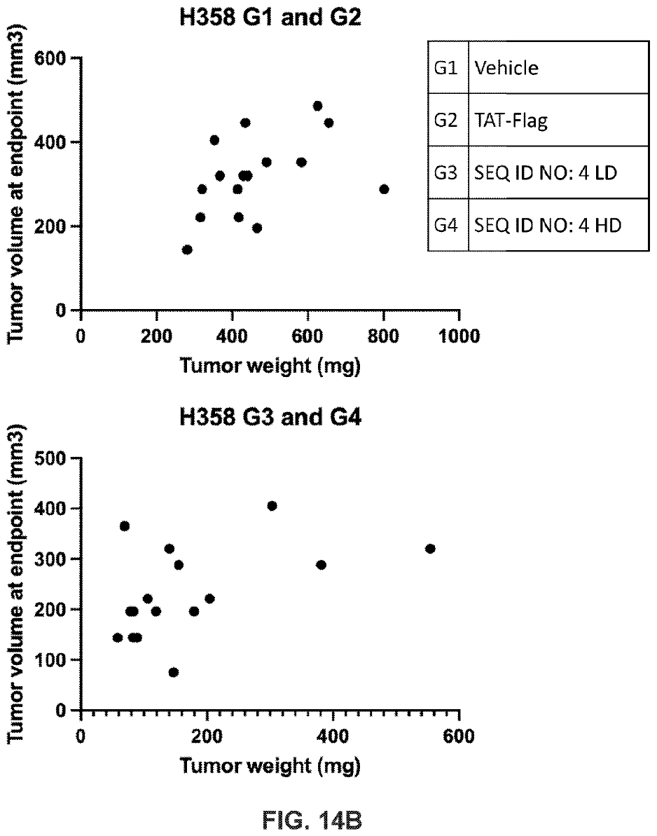

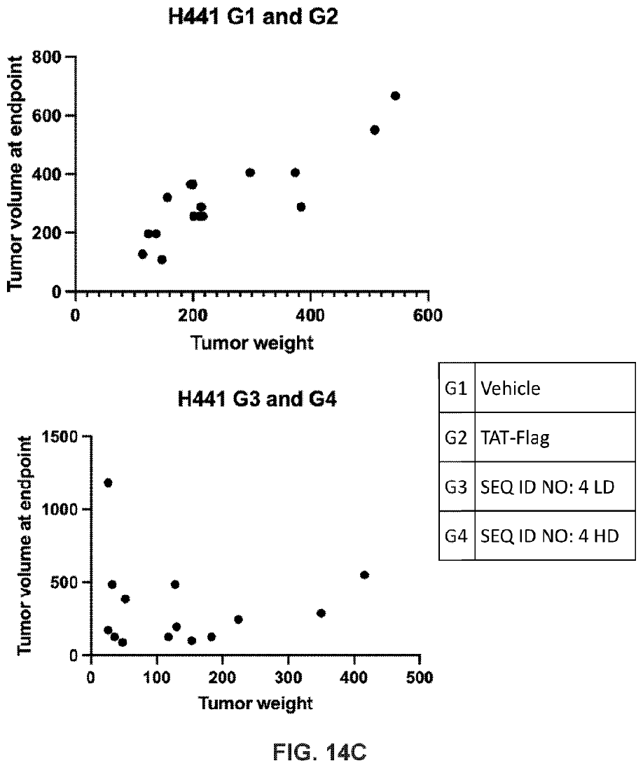

FIGS. 14A-14C show tumor volume by tumor weight correlations for the SW480 model (FIG. 14A), the NCI-H358 model (FIG. 14B), and the NCI-H441 model (FIG. 14C). The correlation demonstrates the difficulty of caliper-based volume measurements with ulceration/scabbing in the H441 model and SW480 model. This measurement difficulty is illustrated for the G3 (SEQ ID NO: 4 peptide low dose) and G4 (SEQ ID NO: 4 peptide high dose) treatment groups. FIGS. 14A-14C show that tumor volumes may not be the most correlative measurement to track anti-tumor efficacy. The control group's tumor weight and volume correlate, however the treated group's tumor weight and volume do not correlate. This is likely due to ulceration and the difficulties of measuring volume by calipers.

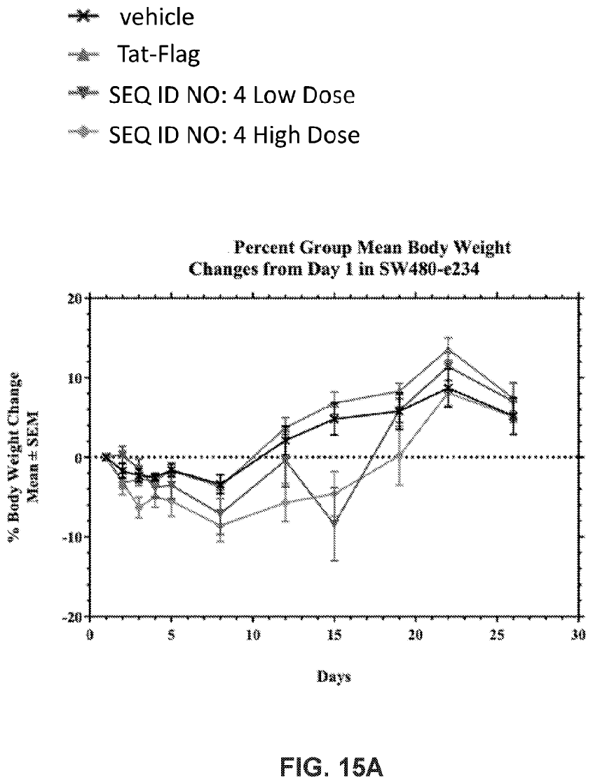

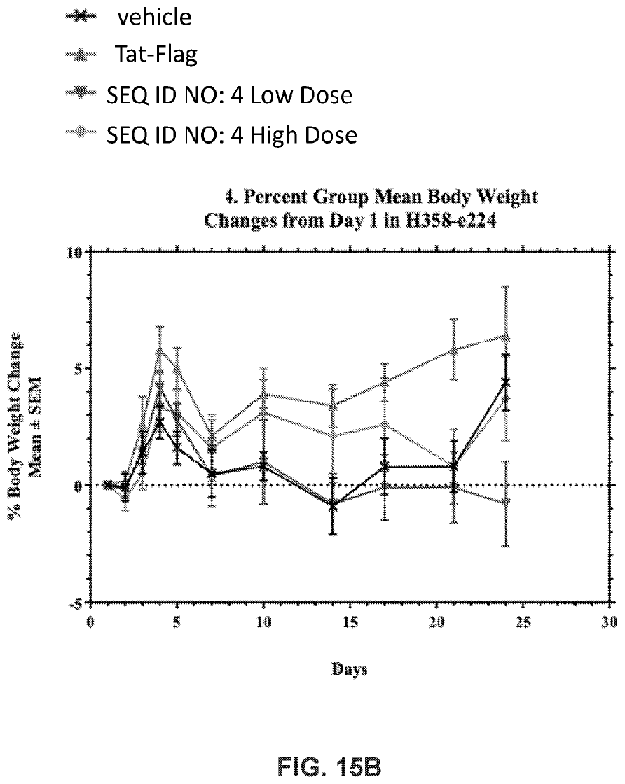

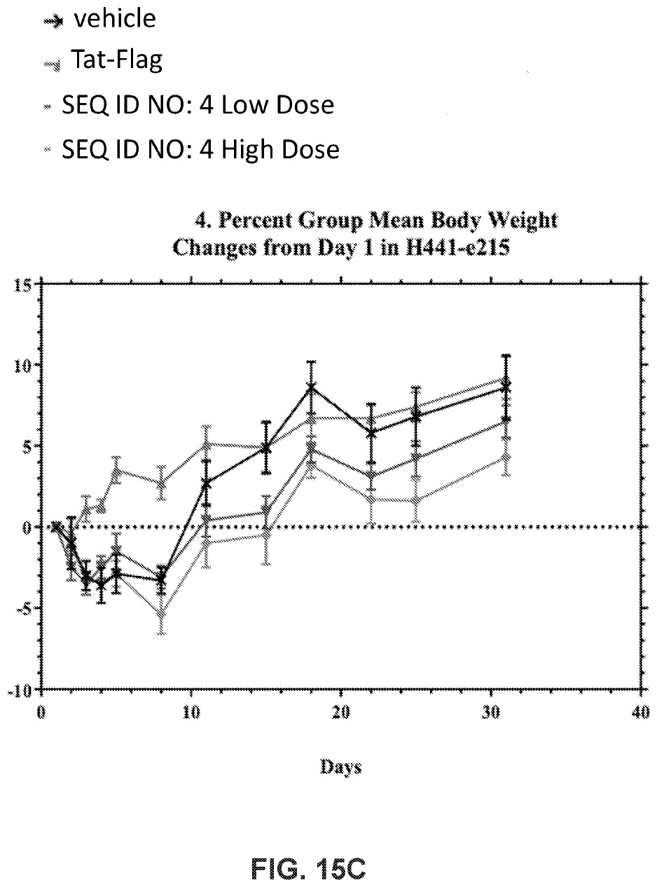

Mean body weights for the groups were recorded throughout the study and are shown in FIGS. 15A-15C for the SW480 (FIG. 15A), NCI-H358 (FIG. 15B), and NCI-H441 (FIG. 15C) treatment groups. The mean body weights were overall comparable between the groups. In the NCI-H358 treatment model, the SEQ ID NO: 4 peptide low dose group showed a decrease in mean body weight at the end of the study as compared to the controls.

Overall, in vivo tumor weight as an endpoint may be a more reliable measurement of efficacy than tumor volume for the H441 model and the SW480 model due to inflammation, ulceration, and scabbing. Several animals in high dose group (SEQ ID NO: 4 peptide-4.5 mg) for the SW480 model were found dead or had to be euthanized but this is likely a model-dependent byproduct of anti-tumor efficacy rather than compound toxicity. In vivo efficacy was observed for the SEQ ID NO: 4 peptide with 3× per week intra-tumoral dosing in the H358 model and SW480 model and shows the peptides of the present disclosure can be used to treat cancers.

### Example 10—Systemically Administered In Vivo Tumor Experiments

This example describes assessment of tumor growth in vivo after administration of SEQ ID NO: 1 peptide, SEQ ID NO: 2 peptide, SEQ ID NO: 3 peptide, SEQ ID NO: 4 peptide, SEQ ID NO: 5 peptide, or SEQ ID NO: 6 peptide an SW480 model and/or a H358 mouse model.

A SW480 and/or NCI-H358 tumor cell line having a G12V KRAS mutation or G12C KRAS mutation in 50% Matrigel are injected subcutaneously in the back of NOD-scid gamma mice. Tumor volumes are allowed to grow and then mice are split into groups with similar tumor size distribution. Mice are systemically administered and repeat-dosed with SEQ ID NO: 1 peptide, SEQ ID NO: 2 peptide, SEQ ID NO: 3 peptide, SEQ ID NO: 4 peptide, SEQ ID NO: 5 peptide, SEQ ID NO: 6 peptide or a control peptide resuspended in PBS at 5 mg/kg, 15 mg/kg, 60 mg/kg, or 100 mg/kg. Tumor growth is measured.

Prior to the systemic study, a pharmacokinetics assessment is completed with a small single dose rodent study. This is used to determine and confirm doses and administration schedules for the systemic in vivo study.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1-40. (canceled)

41. An engineered peptide having at least about 84% sequence identity to SEQ ID NO: 1, SEQ ID NO:2, or SEQ ID NO:3.

42. The engineered peptide of claim 41, wherein the engineered peptide has a length of from about 10 amino acids to about 40 amino acids.

43. The engineered peptide of claim 41 having SEQ ID NO:1, SEQ ID NO:2, or SEQ ID NO:3.

44. The engineered peptide of claim 41, wherein the engineered peptide has an alpha helical structure at a physiological salt concentration and a physiological temperature.

45. The engineered peptide of claim 41, wherein the engineered peptide is a stapled peptide.

46. The engineered peptide of claim 45, wherein the stapled peptide is a (i, i+3) stapled peptide or a (i, i+7) stapled peptide.

47. The engineered peptide of claim 46, wherein the (i) residue of the stapled peptide is R-octenylalanine, S-octenylalanine, R-pentylalanine, or S-pentenylalanine; and wherein the (i+3) residue or the (i+7) residue of the stapled peptide is R-octenylalanine, S-octenylalanine, R-pentylalanine, or S-pentenylalanine.

48. The engineered peptide of claim 47, wherein the (i) residue of the stapled peptide is at amino acid 3 or amino acid 4 of SEQ ID NO:1, SEQ ID NO:2, or SEQ ID NO:3.

49. The engineered peptide of claim 48, wherein the (i) residue of the stapled peptide is R-octenylalanine and the (i+3) residue of the stapled peptide is S-pentenylalanine; or the (i) residue of the stapled peptide is R-octenylalanine and the (i+7) residue of the stapled peptide is S-pentenylalanine.

50. The engineered peptide of claim 48, wherein the engineered peptide is the (i, i+7) stapled peptide, and wherein the (i, i+7) stapled peptide of SEQ ID NO: 1 has at least 84% sequence identity to SEQ ID NO:4; wherein the (i, i+7) stapled peptide of SEQ ID NO:2 has at least 84% sequence identity to SEQ ID NO:5; and wherein the (i, i+7) stapled peptide of SEQ ID NO: 3 has at least 84% sequence identity to SEQ ID NO:6.

51. The engineered peptide of claim 48, wherein the engineered peptide is the (i, i+7) stapled peptide, and wherein the (i, i+7) stapled peptide of SEQ ID NO:1 has SEQ ID NO:4; wherein the (i, i+7) stapled peptide of SEQ ID NO:2 has SEQ ID NO:5; and wherein the (i, i+7) stapled peptide of SEQ ID NO:3 has SEQ ID NO:6.

52. An engineered peptide or a salt thereof comprising at least 85% sequence identity to a contiguous portion of an RB1 peptide, a CHEK2 peptide, an MDM2 peptide, a RAF1 peptide, an RRAS2 peptide, a RAC1 peptide, a PIK3Ca peptide, a PIK3R1 peptide, a VHL peptide, a DDX3X peptide, or a CTNNB1 peptide; wherein the contiguous portion comprises from about 10 amino acids to about 20 amino acids.

53. The engineered peptide of claim 52, wherein the salt of the engineered peptide is an acetate salt or a trifluoroacetate salt.

54. The engineered peptide of claim 52, wherein the engineered peptide is a stapled peptide; and wherein the stapled peptide is an (i, i+3) stapled peptide or an (i, i+7) stapled peptide.

55. A pharmaceutical composition that comprises the engineered peptide of claim 41 and a pharmaceutically acceptable excipient, diluent, or carrier.

56. A method of treating cancer in a subject in need thereof, the method comprising administering to the subject the engineered peptide of claim 41, thereby treating cancer in the subject.

57. The method of claim 56, wherein the cancer comprises a KRAS mutation.

58. The method of claim 56, wherein the cancer is melanoma, colorectal cancer, pancreatic cancer, bladder cancer, breast cancer, ovarian cancer, juvenile myelomonocytic leukemia, a hematologic malignancy, cholangiocarcinoma, uterine cancer, cervical cancer, testicular cancer, or lung cancer.

59. A method of inhibiting cell proliferation in a cancerous cell, the method comprising administering to the cancerous cell the engineered peptide of claim 41, thereby inhibiting proliferation of the cancerous cell.

60. A method of making engineered therapeutic peptides for treatment of cancer, the method comprising:
(a) selecting portions of a candidate polypeptide that display cellular toxicity, as determined in an in vitro assay;
(b) curating the portions of the candidate polypeptides to select a subset of the portions of the candidate polypeptide that adopt an alpha helical structure, as determined by x-ray crystallography;
(c) modifying the subset using mutagenesis to generate engineered therapeutic peptides, and
(d) selecting engineered therapeutic peptides having an IC₅₀ of from about 1 nM to about 50 μM against a cancer cell line, as determined in an in vitro assay.
